(19) United States
(12) Patent Application Publication
(10) Pub. No.: US 2008/0227709 A1
(43) Pub. Date: Sep. 18, 2008

(54) GENE EXPRESSION IN PERIPHERAL BLOOD MONONUCLEAR CELLS FROM CHILDREN WITH DIABETES

(75) Inventors: Maria Virginia Pascual, Dallas, TX (US); Jacques F. Banchereau, Dallas, TX (US); Damien J. Chaussabel, Richardson, TX (US); Ellen Kaizer, Dallas, TX (US); Perrin C. White, Dallas, TX (US)

Correspondence Address: CHALKER FLORES, LLP, 2711 LBJ FRWY, Suite 1036, DALLAS, TX, 75234, US

(73) Assignees: BAYLOR RESEARCH INSTITUTE, Dallas, TX (US); BOARD OF REGENTS, THE UNIVERSITY OF TEXAS SYSTEM, Austin, TX (US)

(21) Appl. No.: 12/046,874

(22) Filed: Mar. 12, 2008

Related U.S. Application Data
(60) Provisional application No. 60/894,784, filed on Mar. 14, 2007.

(51) Int. Cl. A61K 38/00; A61K 31/70; C12Q 1/68; C12Q 1/02; A61P 3/10; C40B 30/04; G01N 33/53

(52) U.S. Cl. 514/12; 514/44; 435/6; 435/29; 506/9; 435/7.92; 702/19

(57) ABSTRACT

The present invention includes composition, methods and systems for detecting, evaluating, diagnosis, tracking and treating Type 1 Diabetes by determining the level of expression of one or more genes listed in Table 1 (e.g., interleukin-1β (IL1B), early growth response gene 3 (EGR3), and prostaglandin-endoperoxide synthase 2 (PTGS2)). The present invention also includes compositions and methods for treating a patient in need thereof with a composition having a therapeutically effective amount of one or more IL-1β antagonists sufficient to spare pancreatic beta cells, including an anti-IL-1β receptor and downstream activators.

### CROSS-REFERENCE TO RELATED APPLICATIONS

This application claims priority to U.S. Provisional Application Ser. No. 60/894,784, filed Mar. 14, 2007, the contents of which is incorporated by reference herein in its entirety.

### TECHNICAL FIELD OF THE INVENTION

The present invention relates in general to the field of diabetes diagnosis, prevention and treatment, and more particularly, to compositions, methods and systems for the detection and use of information obtained from gene expression in peripheral blood mononuclear cells from children with diabetes.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with gene expression array analysis.

Type 1 diabetes (T1D) results from autoimmune destruction of insulin-producing pancreatic beta cells in the Islets of Langerhans (1, 2). This process presumably begins with activation of cellular immunity against self antigens on beta cells, which likely requires genetic susceptibility combined with one or more environmental insults such as a viral infection. Inflammation (insulitis) then occurs, with invasion of islets by immune effector cells and elaboration of cytokines (3-7). Cytokines such as interleukin-1β (IL-1β, the product of the IL1B gene), recruit additional inflammatory cells to the islets and also have direct cytotoxic effects on beta cells (8). Both inflammation and autoimmune recognition are probably required for efficient destruction of beta cells (9, 10). Diabetes becomes clinically apparent when approximately 90% of beta cell mass has been lost (11).

Developing disease-modifying treatments for T1D will require identification of suitable drug targets and markers of therapeutic efficacy. This will require knowledge of changes in gene expression both in pancreatic beta cells and in immune effector cells. It is difficult to obtain pancreas samples from humans with new-onset T1D because the death rate with proper management is extremely low (˜0.1% in our institution (12)). However, islet-infiltrating immune effectors are presumably in equilibrium with circulating pools and may thus be sampled in peripheral blood mononuclear cells (PBMCs). Moreover, metabolic derangements associated with diabetes potentially affect all cells in the body and the resulting changes in gene expression may be sampled in PBMCs.

### SUMMARY OF THE INVENTION

The present invention includes a method for diagnosing, preventing or treating a subject suspected of having Type I diabetes by determining the level of gene expression in peripheral blood mononuclear cells of one or more genes or biomarkers from the group of genes in Table 1; and providing the subject with IL-1β antagonists if the subject have elevated levels of IL-1β gene expression. Examples of IL-1β antagonists include, e.g., anakinra, an anti-IL-1β siRNA, anti-IL-1β and combinations thereof. The IL-1β antagonist may be encapsulated in a capsule, caplet, softgel, gelcap, suppository, film, granule, gum, insert, pastille, pellet, troche, lozenge, disk, poultice or wafer. The IL-1β antagonist may be prepared into a pharmaceutical composition adapted for administration via parenteral, intravenous, oral, intramuscular, intraaortal, intrahepatic, intragastric, intranasal, intrapulmonary, intraperitoneal, subcutaneous, rectal, vaginal, intraosseal or dermal delivery.

Yet another embodiment of the present invention includes a method of identifying a human subject suspected of having diabetes comprising determining the expression level of a biomarker that include one or more of the following genes: interleukin-1β (IL1B), early growth response gene 3 (EGR3), prostaglandin-endoperoxide synthase 2 (PTGS2) and combinations thereof. The method may also include the step of determining expression levels is performed by measuring amounts of mRNA, protein and combinations thereof and/or determining expression levels is performed using hybridization of nucleic acids on a solid support, an oligonucleotide array, sequencing and combinations thereof, and/or the step of determining expression levels is performed using cDNA which is made using mRNA collected from the human cells as a template.

The genes may be detected at the comprises mRNA level and is quantitated by a method selected from the group consisting of polymerase chain reaction, real time polymerase chain reaction, reverse transcriptase polymerase chain reaction, hybridization, probe hybridization, and gene expression array. The step of determining the level of expression is accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing. The sample obtained from a peripheral blood mononuclear cell.

A method of identifying a human subject suspected of having Type 1 diabetes by determining the expression level of a biomarker comprising one or more of the following genes: interleukin-1β (IL1B), early growth response gene 3 (EGR3), and prostaglandin-endoperoxide synthase 2 (PTGS2).

The present invention also includes a computer implemented method for determining a Type 1 diabetes phenotype from a patient suspected of having diabetes by determining the level of expression of one or more genes listed in Table 1, e.g., interleukin-1β (IL1B), early growth response gene 3 (EGR3), and prostaglandin-endoperoxide synthase 2 (PTGS2) combinations thereof and diagnosing the Type 1 diabetes based upon an increase in the probe intensities for the one or more genes as compared to normal gene expression, expression of genes from a non-Type 1 diabetic patient, a Type 3 diabetic patient and combinations thereof.

The present invention also includes a computer readable medium that includes computer-executable instructions in a system for performing the method for diagnosing a patient with Type 1 diabetes by diagnosing Type 1 diabetes based upon the sample probe intensities for six or more genes selected those genes listed in Table 1 and combinations thereof; and calculating a linear correlation coefficient between the sample probe intensities and reference probe intensities; and accepting the tentative diagnosis of Type 1 diabetes if the linear correlation coefficient is greater than a threshold value. In one example the system includes, e.g., determining the level of gene expression of interleukin-1β (IL1B), early growth response gene 3 (EGR3), and prostaglandin-endoperoxide synthase 2 (PTGS2) and combinations thereof in peripheral blood mononuclear cells.

### BRIEF DESCRIPTION OF THE DRAWINGS

For a more complete understanding of the features and advantages of the present invention, reference is now made to the detailed description of the invention along with the accompanying figures and in which:

FIG. 1A, Heat map representing 23 gene probes differentially expressed with a Bonferroni-corrected p<0.05 when comparing newly diagnosed type 1 diabetes (T1D) patients to healthy controls. Each row represents a separate probe set and each column a separate patient sample. IL1B is represented by two probe sets. Each pixel is colored from red (5-fold over-expressed) through yellow (equal) to blue (5-fold under-expressed) compared with median of healthy controls. The uncorrected p value for each comparison and the fold change (median) are listed to the right of the panel. FIG. 1B, Expression levels of the same gene probes are illustrated in T1D patients at 1 and 4 months after diagnosis and in T2D patients.

FIG. 2. RT-PCR results of EGR2 and IL1B were correlated to Genespring generated results for 14 T1D, 7 Healthy, and 3 T2D patients using delta C_{T} results of RT-PCR and the negative logarithm of normalized Genespring values. Spearman r values were: EGR2, 0.91; IL1B, 0.94 (p<0.0001 for both); EGR3, 0.77; FOSB, 0.61; PTGS2, 0.82; SGK, 0.73 (graphs not shown).

FIG. 3. Network of genes with altered expression in T1D. Solid lines represent proteins that are known to physically interact whereas broken lines denote indirect relationships. Red and green objects denote genes that are overexpressed or underexpressed, respectively, in T1D patients at diagnosis, relative to healthy volunteers. Grey genes differ in expression levels between T1D patients and healthy volunteers at uncorrected p values<0.05, but not at false discovery rates (FDR)<0.05. Genes are positioned to represent their function and site of action within a cell. Ig, immunoglobulins; TMRs, transmembrane receptors; GPCRs, G-protein coupled receptors.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “array” refers to a solid support or substrate with one or more peptides or nucleic acid probes attached to the support. Arrays typically have one or more different nucleic acid or peptide probes that are coupled to a surface of a substrate in different, known locations. These arrays, also described as “microarrays” or “gene-chips” that may have 10,000; 20,000, 30,000; or 40,000 different identifiable genes based on the known genome, e.g., the human genome. These pan-arrays are used to detect the entire “transcriptome” or transcriptional pool of genes that are expressed or found in a sample, e.g., nucleic acids that are expressed as RNA, mRNA and the like that may be subjected to RT and/or RT-PCR to made a complementary set of DNA replicons. Arrays may be produced using mechanical synthesis methods, light directed synthesis methods and the like that incorporate a combination of non-lithographic and/or photolithographic methods and solid phase synthesis methods.

Various techniques for the synthesis of these nucleic acid arrays have been described, e.g., fabricated on a surface of virtually any shape or even a multiplicity of surfaces. Arrays may be peptides or nucleic acids on beads, gels, polymeric surfaces, fibers such as fiber optics, glass or any other appropriate substrate. Arrays may be packaged in such a manner as to allow for diagnostics or other manipulation of an all inclusive device, see for example, U.S. Pat. No. 6,955,788, relevant portions incorporated herein by reference.

As used herein, the term “disease” refers to a physiological state of an organism with any abnormal biological state of a cell. Disease includes, but is not limited to, an interruption, cessation or disorder of cells, tissues, body functions, systems or organs that may be inherent, inherited, caused by an infection, caused by abnormal cell function, abnormal cell division and the like. A disease that leads to a “disease state” is generally detrimental to the biological system, that is, the host of the disease. With respect to the present invention, any biological state, such as an infection (e.g., viral, bacterial, fungal, helminthic, etc.), inflammation, autoinflammation, autoimmunity, anaphylaxis, allergies, premalignancy, malignancy, surgical, transplantation, physiological, and the like that is associated with a disease or disorder is considered to be a disease state. A pathological state is generally the equivalent of a disease state.

Disease states may also be categorized into different levels of disease state. As used herein, the level of a disease or disease state is an arbitrary measure reflecting the progression of a disease or disease state as well as the physiological response upon, during and after treatment. Generally, a disease or disease state will progress through levels or stages, wherein the affects of the disease become increasingly severe. The level of a disease state may be impacted by the physiological state of cells in the sample.

As used herein, the terms “therapy” or “therapeutic regimen” refer to those medical steps taken to alleviate or alter a disease state, e.g., a course of treatment intended to reduce or eliminate the affects or symptoms of a disease using pharmacological, surgical, dietary and/or other techniques. A therapeutic regimen may include a prescribed dosage of one or more drugs or surgery. Therapies will most often be beneficial and reduce the disease state but in many instances the effect of a therapy will have non-desirable or side-effects. The effect of therapy will also be impacted by the physiological state of the host, e.g., age, gender, genetics, weight, other disease conditions, etc.

As used herein, the term “pharmacological state” or “pharmacological status” refers to those samples that will be, are and/or were treated with one or more drugs, surgery and the like that may affect the pharmacological state of one or more nucleic acids in a sample, e.g., newly transcribed, stabilized and/or destabilized as a result of the pharmacological intervention. The pharmacological state of a sample relates to changes in the biological status before, during and/or after drug treatment and may serve a diagnostic or prognostic function, as taught herein. Some changes following drug treatment or surgery may be relevant to the disease state and/or may be unrelated side-effects of the therapy. Changes in the pharmacological state are the likely results of the duration of therapy, types and doses of drugs prescribed, degree of compliance with a given course of therapy, and/or un-prescribed drugs ingested.

As used herein, the terms “transcriptional upregulation,” “overexpression, and “overexpressed” refers to an increase in synthesis of RNA by an RNA polymerases using a DNA template in vivo. For example, when used in reference to the methods of the present invention, the term “transcriptional upregulation” refers to an increase of about 1 fold, 2 fold, 2 to 3 fold, 3 to 10 fold, and even greater than 10 fold, in the quantity of mRNA corresponding to a gene of interest detected in a sample derived from an individual predisposed to Type 1 Diabetes as compared to that detected in a sample derived from an individual who is not predisposed to Type 1 Diabetes. However, the system and evaluation is sufficiently specific to require less that a 2 fold change in expression to be detected. Furthermore, the change in expression may be at the cellular level (change in expression within a single cell or cell populations) or may even be evaluated at a tissue level, where there is a change in the number of cells that are expressing the gene. Changes of gene expression in the context of the analysis of a tissue can be due to either regulation of gene activity or relative change in cellular composition. Particularly useful differences are those that are statistically significant.

Conversely, the terms “transcriptional downregulation,” “underexpression” and “underexpressed” are used interchangeably and refer to a decrease in synthesis of RNA, by RNA polymerases using a DNA template. For example, when used in reference to the methods of the present invention, the term “transcriptional downregulation” refers to a decrease of least 1 fold, 2 fold, 2 to 3 fold, 3 to 10 fold, and even greater than 10 fold, in the quantity of mRNA corresponding to a gene of interest detected in a sample derived from an individual predisposed to Type 1 Diabetes as compared to that detected in a sample derived from an individual who is not predisposed to such a condition or to a database of information for wild-type and/or normal control, e.g., Type 2 Diabetes. Again, the system and evaluation is sufficiently specific to require less that a 2 fold change in expression to be detected. Particularly useful differences are those that are statistically significant.

Both transcriptional upregulation/overexpression and transcriptional downregulation/underexpression may also be indirectly monitored through measurement of the translation product or protein level corresponding to the gene of interest. The present invention is not limited to any given mechanism related to upregulation or downregulation of transcription.

The IL-1β antagonist may be administered, e.g., parenterally, intraperitoneally, intraspinally, intravenously, intramuscularly, intravaginally, subcutaneously, or intracerebrally. Dispersions may be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable delivery of the IL-1β antagonist include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi. The carrier may be a solvent or dispersion medium containing, for example, water, ethanol, poly-ol (for example, glycerol, propylene glycol, and liquid polyethylene glycol, and the like), suitable mixtures thereof, and vegetable oils.

The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin.

Sterile injectable solutions may be prepared by incorporating the therapeutic IL-1β antagonist in the required amount in an appropriate solvent with one or a combination of ingredients enumerated above, as required, followed by filtered sterilization. Generally, dispersions are prepared by incorporating the therapeutic compound into a sterile carrier that contains a basic dispersion medium and the required other ingredients from those enumerated above. In the case of sterile powders for the preparation of sterile injectable solutions, the methods of preparation may include vacuum drying, spray drying, spray freezing and freeze-drying that yields a powder of the active ingredient (i.e., the therapeutic compound) plus any additional desired ingredient from a previously sterile-filtered solution thereof.

The IL-1β antagonist may be orally administered, for example, with an inert diluent or an assimilable edible carrier. The therapeutic compound and other ingredients may also be enclosed in a hard or soft shell gelatin capsule, compressed into tablets, or incorporated directly into the subject's diet. For oral therapeutic administration, the therapeutic compound may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied as will be known to the skilled artisan. The amount of the therapeutic compound in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical carrier. The specification for the dosage unit forms of the invention are dictated by and directly dependent on, e.g., (a) the unique characteristics of the therapeutic compound and the particular therapeutic effect to be achieved, and (b) the limitations inherent in the art of compounding such a therapeutic compound for the treatment of a selected condition in a subject.

The present inventors have found that type 1 diabetes (T1D) is accompanied by changes in gene expression in peripheral blood mononuclear cells due to dysregulation of adaptive and innate immunity, counterregulatory responses to immune dysregulation, insulin deficiency and hyperglycemia. Microarray analysis identified 282 genes differing in expression between newly-diagnosed T1D patients and controls at a false discovery rate of 0.05. Changes in expression of interleukin-1β (IL1B), early growth response gene 3 (EGR3), and prostaglandin-endoperoxide synthase 2 (PTGS2) resolved within four months of insulin therapy and were also observed in patients with newly diagnosed type 2 diabetes (T2D) suggesting that they resulted from hyperglycemia. With use of a knowledge base, 81/282 genes could be placed within a network of interrelated genes with predicted functions including apoptosis and cell proliferation. IL1B and the MYC oncogene were the most highly-connected genes in the network. Whereas IL1B was highly overexpressed in both T1D and T2D, MYC was dysregulated only in T1D. Genes associated with proliferation were more likely to be connected to IL1B whereas genes associated with apoptosis were equally likely to be connected to IL1B or MYC. T1D and T2D likely share a final common pathway for beta cell dysfunction that includes secretion of interleukin-1β and prostaglandins by immune effector cells, exacerbating existing beta cell dysfunction, and causing further hyperglycemia. The results identify several targets for disease-modifying therapy of T1D and potential biomarkers for monitoring treatment efficacy.

Microarray techniques were used to identify changes in gene expression in PBMCs from children with new onset T1D. We observed the time course of resolution of such changes with insulin treatment, and determined which of these changes were also found in children with poorly controlled Type 2 diabetes (T2D), in which autoimmunity plays a much less prominent role. These studies identified changes in gene expression in PBMCs that distinguish T1D and T2D, as well as marked changes that are common to both forms of diabetes.

Study population. Peripheral blood mononuclear cells (PBMCs) and serum samples were isolated from 24 healthy volunteers, 43 newly diagnosed T1D patients and 12 newly diagnosed T2D patients (Table 1). We also collected blood samples one and four months after diagnosis from the last 20 of the T1D patients at their routine outpatient visits. For each time point one sample did not pass quality control and was dropped from the analysis. T1D and T2D were distinguished on the basis of age, body habitus, presence or absence of acanthosis nigricans and family history of type 2 diabetes, and presence or absence of autoantibodies to insulin, protein tyrosine phosphatase receptor type N (IA-2, PTPRN) and glutamic acid decarboxylase (GAD65). We allowed low titers of insulin antibodies in T2D patients, which have been previously reported (13). One newly diagnosed teenager with putative T1D was excluded from the study because he was negative for all three antibodies. One putative T2D patient was excluded when she was found to be positive for both IA-2 and GAD.

TABLE 1
312 gene probes (282 unique genes) had an FDR of <0.05 when comparing
newly diagnosed T1D patients to healthy controls. Normalized expression values are listed for
newly diagnosed T1D and healthy controls as well as for T1D patients 1 and 4 months after
diagnosis and for newly-diagnosed T2D patients.

| | | | | | | | | Present |
|---|---|---|---|---|---|---|---|---|
| | | | | T1D, 1 | T1D, 4 | | | in |
| | | | | Month | Month | | | Ingenuity |
| Systematic | FDR | Healthy | T1D New | follow-up | follow-up | T2D | Gene | Pathway |
| 223940_x_at | 0.0498 | 1.363353 | 3.39906 | 1.463348 | 1.299428 | 0.702194 | MALAT-1 | |
| 238908_at | 0.0497 | 0.911523 | 0.622674 | 0.507283 | 0.502404 | 0.703508 | CALU | |
| 241692_at | 0.0497 | 0.941999 | 0.703976 | 0.658092 | 0.61394 | 0.851306 | HNRPLL | |
| 243768_at | 0.0497 | 1.038742 | 0.769039 | 0.834169 | 0.849731 | 1.128139 | SENP6 | |
| 205147_x_at | 0.0497 | 0.996846 | 1.323539 | 1.093569 | 1.05796 | 1.39555 | NCF4 | |
| 207008_at | 0.0492 | 1.086503 | 1.982586 | 1.444336 | 1.230856 | 1.611223 | IL8RB | ✓ |
| 230529_at | 0.0492 | 1.008836 | 0.831476 | 0.880598 | 0.780802 | 1.038305 | HECA | |
| 242858_at | 0.0492 | 0.992682 | 0.762488 | 0.839281 | 0.798004 | 0.781713 | C14orf2 | |
| 234884_x_at | 0.0492 | 1.007387 | 1.469327 | 1.03265 | 1.484244 | 1.056954 | IGLC2 | |
| 220646_s_at | 0.0492 | 0.927558 | 0.504469 | 0.546674 | 0.613675 | 0.853097 | KLRF1 | |
| 216682_s_at | 0.0487 | 1.098977 | 0.719404 | 0.956118 | 1.129677 | 0.975998 | P38IP | |
| 237181_at | 0.0487 | 1.035286 | 0.764448 | 0.94573 | 0.922479 | 0.822278 | PPP2R5C | |
| 202810_at | 0.0484 | 0.988227 | 0.872303 | 0.980991 | 0.953917 | 1.034228 | DRG1 | |
| 213593_s_at | 0.0484 | 1.08201 | 0.811101 | 0.929895 | 0.834722 | 0.71784 | TRA2A | |
| 208774_at | 0.0481 | 1.007206 | 1.387784 | 1.410861 | 1.447677 | 1.193977 | CSNK1D | ✓ |
| 230535_s_at | 0.0481 | 0.918442 | 0.633242 | 0.920633 | 0.791582 | 1.101614 | TUBB1 | |
| 242492_at | 0.0477 | 1.025379 | 0.769541 | 0.981963 | 1.037144 | 0.893252 | CLNS1A | |
| 211881_x_at | 0.0475 | 0.974695 | 1.390196 | 1.000421 | 1.270499 | 1.05704 | IGLJ3 | |
| 204976_s_at | 0.0470 | 0.977491 | 0.797105 | 0.953966 | 0.928253 | 1.097668 | AMMECR1 | |
| 209082_s_at | 0.0469 | 1.021274 | 1.356565 | 1.141 | 1.276822 | 0.964197 | COL18A1 | ✓ |
| 217845_x_at | 0.0464 | 0.993311 | 0.80923 | 0.887384 | 0.964488 | 1.050742 | HIGD1A | |
| 203414_at | 0.0463 | 0.881669 | 0.61528 | 0.705223 | 0.699352 | 0.789474 | MMD | |
| 213684_s_at | 0.0461 | 1.101852 | 0.719137 | 0.942749 | 0.936118 | 1.00314 | LIM | ✓ |
| 223147_s_at | 0.0461 | 1.06033 | 1.465088 | 1.219447 | 1.274929 | 1.036303 | WDR33 | |
| 220052_s_at | 0.0459 | 1.059263 | 1.381241 | 1.179961 | 1.236511 | 1.335935 | TINF2 | ✓ |
| 226077_at | 0.0446 | 0.947498 | 1.255226 | 0.989991 | 0.973909 | 1.20453 | FLJ31951 | |
| 210024_s_at | 0.0446 | 1.00366 | 0.837473 | 0.890586 | 0.85444 | 0.843063 | UBE2E3 | |
| 201392_s_at | 0.0442 | 1.053899 | 1.334403 | 1.034939 | 0.948959 | 1.380742 | IGF2R | |
| 239049_at | 0.0438 | 1.033839 | 0.766037 | 0.720443 | 0.694986 | 0.779814 | | |
| 208697_s_at | 0.0438 | 0.980631 | 0.842984 | 1.005368 | 1.012023 | 1.024368 | EIF3S6 | |
| 231106_at | 0.0435 | 1.025548 | 0.842553 | 0.880782 | 0.965641 | 1.008777 | LOC255326 | |
| 241751_at | 0.0434 | 1.017255 | 0.763999 | 0.881289 | 0.858019 | 0.913149 | OFD1 | |
| 230868_at | 0.0434 | 0.987116 | 0.733456 | 0.83901 | 0.857622 | 0.803324 | HIAT1 | |
| 217739_s_at | 0.0431 | 1.004358 | 1.945946 | 0.903613 | 0.600306 | 1.812722 | PBEF1 | |
| 224327_s_at | 0.0427 | 0.944037 | 1.5144 | 1.108841 | 1.015915 | 1.232647 | DGAT2 | |
| 210484_s_at | 0.0427 | 0.706332 | 1.4849 | 0.932496 | 0.624225 | 0.834965 | TNFRSF10C | |
| 223046_at | 0.0427 | 0.972865 | 1.208905 | 0.988457 | 1.044866 | 1.170877 | EGLN1 | ✓ |
| 203198_at | 0.0427 | 0.946441 | 1.246439 | 0.675256 | 0.609355 | 0.63388 | CDK9 | ✓ |
| 211662_s_at | 0.0423 | 1.001891 | 0.879862 | 0.929971 | 0.920098 | 0.966985 | VDAC2 | ✓ |
| 230185_at | 0.0419 | 0.971946 | 1.196263 | 1.125986 | 1.181908 | 1.309545 | THAP9 | |
| 229967_at | 0.0408 | 1.061572 | 2.130441 | 1.492791 | 1.274976 | 2.133424 | CKLFSF2 | |
| 242438_at | 0.0407 | 1.005338 | 0.834434 | 0.846502 | 0.786264 | 1.017121 | ASXL1 | |
| 223265_at | 0.0407 | 0.948348 | 1.367733 | 1.160239 | 1.348674 | 0.722968 | SH3BP5L | |
| 232216_at | 0.0404 | 0.984754 | 0.687209 | 0.64591 | 0.557223 | 0.759283 | YME1L1 | ✓ |
| 226275_at | 0.0402 | 1.087224 | 1.591003 | 1.165967 | 1.063232 | 1.588966 | MAD | |
| 244803_at | 0.0402 | 0.943561 | 0.670472 | 0.751306 | 0.693161 | 0.86086 | | |
| 203066_at | 0.0397 | 1.003763 | 1.322627 | 1.220769 | 1.140396 | 1.513221 | GALNAC4S-6ST | |
| 213598_at | 0.0395 | 1.025175 | 0.830283 | 0.930569 | 0.956208 | 1.025977 | HSA9761 | |
| 232521_at | 0.0387 | 0.974771 | 0.728149 | 0.804742 | 0.959636 | 0.861112 | PCSK7 | |
| 244354_at | 0.0385 | 1.027952 | 0.793331 | 1.033049 | 1.04988 | 1.054802 | | |
| 216401_x_at | 0.0385 | 0.922503 | 1.503036 | 0.858654 | 1.377942 | 0.84475 | IGKC | |
| 227251_at | 0.0385 | 1.015684 | 0.851413 | 0.974719 | 1.099593 | 1.004218 | WDR22 | |
| 235242_at | 0.0385 | 1.041403 | 0.822332 | 0.941866 | 0.857908 | 1.17964 | | |
| 205844_at | 0.0379 | 1.011231 | 1.68468 | 1.347524 | 1.432101 | 2.223076 | VNN1 | |
| 215203_at | 0.0379 | 1.10498 | 0.737621 | 0.910453 | 1.011948 | 0.894413 | GOLGA4 | |
| 214011_s_at | 0.0379 | 0.997356 | 1.218728 | 1.309777 | 1.289319 | 1.136465 | HSPC111 | |
| 204882_at | 0.0376 | 1.085486 | 1.473255 | 1.552252 | 1.655045 | 1.409373 | ARHGAP25 | |
| 223200_s_at | 0.0376 | 1.038209 | 1.393335 | 1.051874 | 1.07248 | 0.975302 | FLJ11301 | |
| 207677_s_at | 0.0376 | 1.043845 | 1.427109 | 1.245901 | 1.08085 | 1.246137 | NCF4 | ✓ |
| 207275_s_at | 0.0376 | 1.111829 | 1.810442 | 1.33652 | 1.157585 | 2.296518 | ACSL1 | ✓ |
| 202859_x_at | 0.0376 | 0.786721 | 2.709688 | 1.110942 | 0.758013 | 2.221863 | IL8 | ✓ |
| 203588_s_at | 0.0376 | 0.968153 | 0.733804 | 0.942768 | 0.84354 | 0.858268 | TFDP2 | ✓ |
| 212000_at | 0.0376 | 1.026141 | 0.826099 | 0.991826 | 0.923545 | 0.950685 | SFRS14 | |
| 216278_at | 0.0376 | 0.998814 | 0.604062 | 0.853314 | 0.719053 | 0.707572 | KIAA0256 | |
| 241425_at | 0.0376 | 0.999569 | 0.758904 | 0.735938 | 0.660895 | 0.941986 | NUPL1 | |
| 224568_x_at | 0.0376 | 1.320035 | 3.421374 | 1.47161 | 1.386858 | 0.666894 | MALAT-1 | |
| 237118_at | 0.0376 | 1.023458 | 0.721438 | 0.660047 | 0.801745 | 0.86233 | ANP32A | |
| 209526_s_at | 0.0376 | 1.091004 | 0.782399 | 0.848617 | 0.828362 | 0.719334 | | |
| 230395_at | 0.0376 | 0.944285 | 0.614125 | 0.682163 | 0.713574 | 0.890519 | DREV1 | |
| 234366_x_at | 0.0375 | 1.010873 | 1.559191 | 1.460598 | 1.718751 | 1.026991 | IGLC2 | |
| 230004_at | 0.0375 | 1.045127 | 0.74255 | 1.13223 | 1.005568 | 1.195664 | USP24 | |
| 225414_at | 0.0374 | 1.035422 | 1.424761 | 0.835388 | 0.821828 | 1.175262 | RNF149 | |
| 236495_at | 0.0374 | 1.071172 | 2.169329 | 0.894083 | 0.724194 | 1.819902 | PBEF1 | |
| 231108_at | 0.0374 | 0.978591 | 0.694286 | 0.731176 | 0.621134 | 0.588171 | | |
| 221840_at | 0.0374 | 0.954525 | 1.247413 | 1.143741 | 1.13555 | 1.379552 | PTPRE | |
| 212722_s_at | 0.0372 | 0.994509 | 1.279735 | 0.991058 | 0.898289 | 1.045019 | PTDSR | |
| 243561_at | 0.0369 | 0.998649 | 0.672402 | 0.817369 | 0.953416 | 0.735158 | YAF2 | ✓ |
| 201540_at | 0.0369 | 1.046501 | 0.75513 | 0.906339 | 1.003023 | 0.874961 | FHL1 | |
| 222437_s_at | 0.0368 | 0.956394 | 0.810276 | 0.855464 | 0.834581 | 0.967927 | VPS24 | |
| 208908_s_at | 0.0363 | 0.965333 | 0.788149 | 0.821529 | 0.829192 | 1.085203 | CAST | |
| 203338_at | 0.0357 | 0.99864 | 0.852229 | 0.83025 | 0.820614 | 0.908424 | PPP2R5E | |
| 203633_at | 0.0355 | 1.064067 | 1.364676 | 1.19262 | 1.251772 | 1.465436 | CPT1A | ✓ |
| 206515_at | 0.0355 | 0.859087 | 1.708633 | 0.912596 | 0.845333 | 1.956985 | CYP4F3 | ✓ |
| 211576_s_at | 0.0352 | 0.916349 | 1.253437 | 1.270848 | 1.146106 | 1.211836 | SLC19A1 | ✓ |
| 210987_x_at | 0.0348 | 0.950558 | 0.763752 | 0.936457 | 0.918583 | 0.93808 | TPM1 | |
| 210119_at | 0.0348 | 0.900934 | 2.013616 | 1.15705 | 0.858026 | 1.891069 | KCNJ15 | |
| 202157_s_at | 0.0348 | 0.964949 | 0.823519 | 0.906364 | 0.92219 | 1.065645 | CUGBP2 | |
| 203591_s_at | 0.0348 | 1.092034 | 1.591224 | 1.311179 | 1.163829 | 1.330385 | CSF3R | ✓ |
| 211908_x_at | 0.0347 | 1.122368 | 1.958046 | 1.166884 | 1.533416 | 1.220641 | IGHG1 | |
| 215379_x_at | 0.0347 | 1.048356 | 1.952508 | 1.169256 | 1.564915 | 1.343552 | IGLJ3 | |
| 209303_at | 0.0347 | 0.985634 | 0.842712 | 0.973467 | 0.977255 | 0.867011 | NDUFS4 | |
| 226333_at | 0.0347 | 0.972634 | 1.26124 | 1.108914 | 1.08077 | 1.214376 | IL6R | |
| 203060_s_at | 0.0347 | 0.954154 | 0.683199 | 0.977323 | 0.69165 | 1.057965 | PAPSS2 | |
| 201163_s_at | 0.0345 | 1.048873 | 0.689451 | 0.911496 | 0.921191 | 0.983939 | IGFBP7 | ✓ |
| 234210_x_at | 0.0345 | 0.984723 | 0.693297 | 0.688835 | 0.605646 | 0.878651 | | |
| 232630_at | 0.0345 | 1.025328 | 0.573005 | 0.843758 | 0.881261 | 1.144546 | MMRP19 | |
| 210986_s_at | 0.0338 | 1.031825 | 0.73003 | 0.964678 | 0.950272 | 0.829836 | TPM1 | ✓ |
| 227762_at | 0.0338 | 1.038881 | 0.713737 | 0.901177 | 0.865919 | 0.863961 | ZNF145 | |
| 229593_at | 0.0338 | 1.006383 | 0.765104 | 0.906021 | 0.86248 | 0.834851 | H2AFY | ✓ |
| 208870_x_at | 0.0338 | 1.001297 | 0.865404 | 1.040431 | 1.073953 | 0.999744 | ATP5C1 | |
| 229434_at | 0.0338 | 0.996675 | 0.773307 | 0.871812 | 0.831998 | 1.154253 | HNRPD | |
| 214784_x_at | 0.0338 | 1.000166 | 1.204025 | 1.137754 | 1.069198 | 1.200257 | XPO6 | |
| 206770_s_at | 0.0337 | 1.039925 | 0.785087 | 0.78521 | 0.808196 | 1.101514 | SLC35A3 | |
| 200798_x_at | 0.0337 | 0.965979 | 1.409602 | 0.943253 | 0.942095 | 1.349478 | MCL1 | |
| 201175_at | 0.0337 | 1.001314 | 1.207241 | 1.18095 | 1.150517 | 1.175283 | TXNDC14 | |
| 243249_at | 0.0332 | 1.040052 | 0.825144 | 0.901977 | 0.931704 | 1.048149 | C14orf119 | |
| 41387_r_at | 0.0332 | 0.969833 | 1.277765 | 1.078283 | 1.060282 | 0.930021 | JMJD3 | |
| 227697_at | 0.0332 | 1.121452 | 2.384171 | 0.852672 | 0.710971 | 1.502212 | SOCS3 | ✓ |
| 228879_at | 0.0329 | 0.945057 | 1.314259 | 1.141172 | 1.152679 | 0.92616 | | |
| 209385_s_at | 0.0328 | 0.917897 | 0.673283 | 0.857211 | 0.850807 | 0.974187 | PROSC | |
| 228376_at | 0.0328 | 0.968563 | 0.668074 | 0.881116 | 0.777176 | 0.961148 | a1/3GTP | |
| 235984_at | 0.0326 | 1.050475 | 0.822341 | 0.879999 | 0.808148 | 0.939732 | ZNF313 | |
| 235556_at | 0.0322 | 1.014561 | 0.852591 | 0.978527 | 0.84957 | 1.002792 | | |
| 216954_x_at | 0.0322 | 1.021689 | 0.835371 | 0.985647 | 0.953977 | 0.781153 | ATP5O | |
| 221766_s_at | 0.0322 | 1.000791 | 0.709592 | 1.007035 | 0.815788 | 1.11682 | C6orf37 | |
| 200665_s_at | 0.0315 | 0.934144 | 0.506217 | 0.707411 | 0.738111 | 0.958147 | SPARC | ✓ |
| 236699_at | 0.0315 | 0.872746 | 0.561117 | 0.630601 | 0.636072 | 0.87649 | MBNL2 | |
| 226153_s_at | 0.0302 | 1.027082 | 0.859316 | 0.897311 | 0.889432 | 1.123349 | CNOT6L | |
| 235983_at | 0.0302 | 1.01012 | 0.796671 | 0.892201 | 0.889679 | 0.864015 | ALS2CR3 | |
| 216557_x_at | 0.03 | 1.077744 | 1.616381 | 1.077329 | 1.476563 | 1.032133 | IGHG1 | |
| 203887_s_at | 0.0299 | 1.009925 | 1.966219 | 1.268705 | 1.080139 | 1.743274 | THBD | ✓ |
| 242349_at | 0.0299 | 1.009164 | 0.81049 | 0.949322 | 0.932348 | 0.827037 | HECTD1 | |
| 219938_s_at | 0.0299 | 0.987107 | 0.717082 | 0.830222 | 0.728393 | 0.910757 | PSTPIP2 | |
| 213995_at | 0.0299 | 0.957004 | 0.792348 | 0.902038 | 0.897271 | 0.903205 | ATP5S | |
| 238706_at | 0.0299 | 1.0479 | 0.726738 | 0.625535 | 0.659847 | 0.993464 | PAPD4 | |
| 200796_s_at | 0.0299 | 0.81494 | 1.6134 | 0.882638 | 0.920608 | 1.017442 | MCL1 | ✓ |
| 227404_s_at | 0.0297 | 0.760108 | 2.217996 | 1.243703 | 0.975313 | 3.051484 | EGR1 | |
| 211746_x_at | 0.0293 | 1.035484 | 0.890748 | 1.00284 | 0.999037 | 1.099539 | PSMA1 | ✓ |
| 214768_x_at | 0.0287 | 1.105826 | 1.769847 | 1.049148 | 1.367852 | 0.872615 | | |
| 211816_x_at | 0.0287 | 0.853645 | 1.435576 | 0.788487 | 0.682175 | 1.024009 | FCAR | |
| 228105_at | 0.0287 | 1.036676 | 0.783027 | 0.852118 | 0.864155 | 1.028855 | C11orf23 | |
| 238913_at | 0.0281 | 0.989083 | 0.70905 | 0.76622 | 0.792108 | 0.710292 | CPSF6 | |
| 241879_at | 0.0281 | 1.034755 | 0.7335 | 1.078535 | 0.99847 | 1.020112 | | |
| 231812_x_at | 0.0277 | 0.961448 | 1.306354 | 1.211513 | 1.302925 | 1.278207 | RNUXA | |
| 205022_s_at | 0.0277 | 1.027865 | 0.810205 | 0.956689 | 0.899327 | 0.814103 | CHES1 | |
| 210993_s_at | 0.0277 | 0.913683 | 0.615834 | 0.946998 | 0.754225 | 1.108006 | SMAD1 | ✓ |
| 212843_at | 0.0277 | 1.065643 | 0.657534 | 0.935914 | 0.901051 | 1.027094 | NCAM1 | ✓ |
| 201693_s_at | 0.0277 | 0.761956 | 1.992585 | 1.11852 | 0.917813 | 2.46243 | EGR1 | |
| 229574_at | 0.026 | 1.056784 | 0.753846 | 0.787856 | 0.755072 | 0.829691 | TRA2A | |
| 229934_at | 0.0255 | 0.898915 | 1.587602 | 1.715653 | 1.60242 | 1.8682 | | |
| 242877_at | 0.0247 | 0.965416 | 0.633215 | 0.595215 | 0.701972 | 0.713209 | C19orf13 | |
| 216542_x_at | 0.024 | 0.971992 | 1.404043 | 1.023935 | 1.042839 | 0.929428 | IGHG1 | |
| 206245_s_at | 0.024 | 1.015357 | 1.282608 | 1.035545 | 1.010402 | 1.224147 | IVNS1ABP | |
| 202822_at | 0.023 | 0.993927 | 0.74549 | 1.074782 | 1.106992 | 1.150837 | LPP | |
| 228008_at | 0.0226 | 1.03325 | 1.288702 | 1.215884 | 1.160234 | 1.138622 | | |
| 201235_s_at | 0.0226 | 0.855178 | 1.514005 | 1.174384 | 1.351371 | 1.267922 | BTG2 | |
| 219110_at | 0.0226 | 1.006412 | 1.183985 | 1.154331 | 1.214279 | 1.014554 | NOLA1 | |
| 228455_at | 0.0226 | 0.991755 | 0.750479 | 0.898291 | 1.070534 | 0.993522 | SLC16A4 | |
| 208686_s_at | 0.0226 | 1.010621 | 1.265238 | 1.111906 | 1.04532 | 0.837633 | BRD2 | ✓ |
| 211163_s_at | 0.0221 | 0.869106 | 2.008078 | 1.011547 | 0.727596 | 1.609339 | TNFRSF10C | |
| 243037_at | 0.0221 | 1.020577 | 0.70985 | 0.638602 | 0.495641 | 0.815817 | FUBP1 | ✓ |
| 242968_at | 0.0216 | 1.006975 | 0.777934 | 0.8108 | 0.816789 | 0.850541 | WHSC1L1 | |
| 215813_s_at | 0.0216 | 0.968077 | 0.720279 | 0.89466 | 0.837868 | 0.947996 | PTGS1 | |
| 204269_at | 0.0216 | 1.0169 | 1.434021 | 1.079973 | 1.00107 | 1.016356 | PIM2 | |
| 209336_at | 0.0212 | 0.976007 | 1.313636 | 1.26169 | 1.073154 | 0.917474 | PWP2H | |
| 209939_x_at | 0.0211 | 0.991492 | 0.802352 | 0.847775 | 0.930274 | 1.104238 | CFLAR | ✓ |
| 235679_at | 0.0211 | 0.989085 | 0.785617 | 1.086461 | 1.066613 | 0.975108 | | |
| 240094_at | 0.0206 | 0.979362 | 0.718204 | 0.803517 | 0.70418 | 1.102802 | DJ971N18.2 | |
| AFFX-r2- | 0.0196 | 1.001167 | 1.648921 | 1.123259 | 1.110089 | 1.06922 | | |
| Hs28SrRNA- | | | | | | | | |
| 5_at | | | | | | | | |
| 224651_at | 0.0194 | 0.956646 | 0.722882 | 0.843131 | 0.781533 | 0.930108 | C10orf9 | |
| 214731_at | 0.0194 | 1.007476 | 0.748558 | 0.997998 | 0.932785 | 0.910209 | CTTNBP2NL | |
| 226022_at | 0.0194 | 0.987503 | 1.488048 | 1.08242 | 0.948622 | 1.397026 | SASH1 | |
| 207798_s_at | 0.0194 | 0.927947 | 0.597525 | 0.589337 | 0.538551 | 0.494842 | ATXN2L | |
| 205099_s_at | 0.0194 | 0.96663 | 1.706206 | 1.448591 | 1.143311 | 1.770972 | CCR1 | |
| 236921_at | 0.0194 | 1.008593 | 0.765737 | 0.887894 | 0.801292 | 0.840048 | EMB | |
| 231165_at | 0.0194 | 1.003329 | 0.595107 | 0.767763 | 0.837665 | 1.025394 | DDHD1 | |
| 205684_s_at | 0.0194 | 1.003507 | 0.79092 | 0.979743 | 0.995696 | 1.058896 | DENND4C | |
| 212742_at | 0.0194 | 1.008846 | 0.847585 | 0.935088 | 0.93266 | 0.965891 | ZNF364 | ✓ |
| 227510_x_at | 0.0194 | 0.954996 | 2.681865 | 1.75285 | 2.217317 | 0.544722 | PRO1073 | |
| 243514_at | 0.0192 | 1.078244 | 0.813221 | 0.817568 | 0.686487 | 0.869929 | WDFY2 | |
| 222311_s_at | 0.019 | 0.975197 | 0.688404 | 0.883813 | 0.897488 | 0.990368 | SFRS15 | |
| 211068_x_at | 0.019 | 0.971695 | 0.859899 | 0.924538 | 0.897512 | 1.020424 | FAM21C | |
| 242109_at | 0.0184 | 0.932229 | 0.609508 | 0.425873 | 0.388648 | 0.484729 | | |
| 220939_s_at | 0.0184 | 0.999409 | 0.841076 | 0.923936 | 0.95513 | 1.183488 | DPP8 | |
| 204108_at | 0.0184 | 1.017202 | 1.245786 | 1.161915 | 1.131599 | 1.130159 | NFYA | ✓ |
| 228325_at | 0.0184 | 1.004505 | 1.52904 | 0.89852 | 0.787106 | 1.361317 | KIAA0146 | |
| 232138_at | 0.0184 | 0.999604 | 0.734164 | 0.793418 | 0.745542 | 1.010467 | MBNL2 | |
| 201695_s_at | 0.0184 | 1.071494 | 1.491268 | 1.635032 | 1.653587 | 1.796414 | NP | ✓ |
| 203105_s_at | 0.0184 | 0.990872 | 0.794554 | 0.901935 | 0.924611 | 1.302084 | DNM1L | |
| 239818_x_at | 0.0184 | 0.708047 | 1.966326 | 0.977036 | 0.797583 | 1.219277 | TRIB1 | |
| 237856_at | 0.0184 | 0.956539 | 0.706623 | 0.894583 | 0.860547 | 0.9648 | RAP1GDS1 | |
| 230703_at | 0.0184 | 1.031296 | 0.669363 | 0.800423 | 0.613284 | 0.815555 | C14orf32 | |
| 215214_at | 0.0181 | 0.996308 | 1.636919 | 1.397556 | 1.699668 | 1.01258 | IGLC2 | ✓ |
| 216621_at | 0.0181 | 1.051422 | 0.689048 | 0.818913 | 0.929655 | 0.919856 | ROCK1 | ✓ |
| 206222_at | 0.0181 | 1.031788 | 1.894518 | 1.192133 | 1.056245 | 1.590383 | TNFRSF10C | |
| 203658_at | 0.0181 | 0.985835 | 1.274624 | 1.147561 | 1.103947 | 1.323086 | SLC25A20 | |
| 205128_x_at | 0.0177 | 0.954145 | 0.703677 | 0.828329 | 0.882044 | 0.961593 | PTGS1 | ✓ |
| 228846_at | 0.0177 | 1.053528 | 1.870718 | 1.141084 | 0.92438 | 1.831064 | MAD | ✓ |
| 242743_at | 0.0172 | 1.037206 | 1.334972 | 1.433561 | 1.457836 | 1.003937 | IL4R | ✓ |
| 218250_s_at | 0.0172 | 1.001075 | 0.840813 | 0.941406 | 0.907309 | 1.038633 | CNOT7 | ✓ |
| 204115_at | 0.0169 | 0.846893 | 0.45709 | 0.568392 | 0.595841 | 0.736674 | GNG11 | ✓ |
| 221571_at | 0.0169 | 1.010583 | 1.310029 | 1.22059 | 1.180414 | 1.121957 | TRAF3 | ✓ |
| 229803_s_at | 0.0167 | 1.007475 | 0.735854 | 1.044848 | 1.040431 | 1.070698 | | |
| 218645_at | 0.0167 | 0.942803 | 0.714669 | 0.808261 | 0.774937 | 0.836524 | ZNF277 | |
| 222662_at | 0.0166 | 1.017965 | 1.559751 | 1.27997 | 1.111935 | 1.449142 | LOC286044 | |
| 217022_s_at | 0.0166 | 1.02909 | 2.049312 | 1.297078 | 1.32098 | 1.129098 | MGC27165 | |
| 229723_at | 0.0159 | 1.012519 | 1.325794 | 1.133633 | 1.117863 | 1.181658 | TAGAP | |
| 201531_at | 0.0159 | 1.005407 | 1.52494 | 1.015045 | 0.934695 | 1.103845 | ZFP36 | ✓ |
| 222670_s_at | 0.0159 | 0.993164 | 1.824372 | 0.981341 | 0.769227 | 1.781366 | MAFB | |
| 201694_s_at | 0.0159 | 0.654797 | 1.78305 | 1.143119 | 0.934794 | 2.031332 | EGR1 | ✓ |
| 214917_at | 0.0159 | 0.974875 | 0.7143 | 0.841413 | 0.843241 | 0.779521 | PRKAA1 | |
| 208803_s_at | 0.0159 | 1.003449 | 0.820692 | 0.950186 | 1.016438 | 1.129951 | SRP72 | |
| 203415_at | 0.0159 | 0.962234 | 1.168577 | 1.250406 | 1.290838 | 1.192712 | PDCD6 | ✓ |
| 239654_at | 0.0159 | 0.963305 | 0.705144 | 0.868072 | 0.903663 | 0.964255 | TSCOT | |
| 205603_s_at | 0.0159 | 0.995527 | 0.778263 | 0.965946 | 0.859117 | 0.933637 | DIAPH2 | ✓ |
| 210176_at | 0.0159 | 1.027185 | 1.569915 | 1.332651 | 1.104354 | 1.811346 | TLR1 | ✓ |
| 211643_x_at | 0.0159 | 1.009605 | 1.519608 | 1.237848 | 1.615528 | 0.972001 | IGKC | |
| 212287_at | 0.0159 | 0.976929 | 0.806968 | 0.902008 | 0.904211 | 0.897486 | JJAZ1 | |
| 212063_at | 0.0159 | 0.979489 | 0.847497 | 0.83193 | 0.843965 | 0.841504 | CD44 | ✓ |
| 236019_at | 0.0159 | 1.006566 | 0.687083 | 0.778719 | 0.711938 | 0.871047 | | |
| 202081_at | 0.0159 | 0.991023 | 1.387281 | 0.975652 | 0.979591 | 1.305892 | IER2 | ✓ |
| 204616_at | 0.0159 | 0.985128 | 0.828295 | 0.840292 | 0.864638 | 0.920087 | UCHL3 | |
| 219253_at | 0.0153 | 0.975863 | 1.313643 | 1.026461 | 1.127687 | 0.85135 | FAM11B | |
| 207808_s_at | 0.0153 | 0.853817 | 0.439433 | 0.75339 | 0.770317 | 0.696055 | PROS1 | ✓ |
| 232629_at | 0.0153 | 0.949032 | 2.029141 | 1.012866 | 0.817135 | 2.805916 | PROK2 | |
| 222465_at | 0.0153 | 1.000641 | 0.782539 | 0.739285 | 0.733434 | 0.952898 | C15orf15 | |
| 202662_s_at | 0.0153 | 0.918615 | 0.644018 | 0.73308 | 0.703615 | 1.033402 | ITPR2 | |
| 212077_at | 0.015 | 1.000662 | 0.576742 | 0.775282 | 0.713565 | 0.860015 | CALD1 | ✓ |
| 201164_s_at | 0.015 | 0.982736 | 0.801137 | 0.910282 | 0.884217 | 1.006008 | PUM1 | |
| 235037_at | 0.015 | 0.971806 | 0.7344 | 0.941781 | 0.916799 | 0.838581 | MGC15397 | |
| 228528_at | 0.0147 | 0.952345 | 1.301443 | 1.455353 | 1.33913 | 1.431278 | | |
| 224939_at | 0.0147 | 1.036122 | 0.827646 | 0.866498 | 0.898493 | 0.944592 | 182-FIP | |
| 224754_at | 0.0147 | 1.008465 | 0.841097 | 0.951757 | 1.038035 | 1.208821 | SP1 | ✓ |
| 217775_s_at | 0.0147 | 0.948531 | 0.709693 | 0.986506 | 0.994069 | 1.142681 | RDH11 | |
| 237626_at | 0.0146 | 1.016095 | 0.619875 | 0.694442 | 0.693349 | 0.860219 | RB1CC1 | |
| 211634_x_at | 0.0146 | 0.978546 | 1.773841 | 0.937101 | 1.471152 | 1.010691 | IGHG1 | ✓ |
| 213366_x_at | 0.0146 | 0.999204 | 0.835363 | 0.983703 | 1.040297 | 1.010819 | ATP5C1 | |
| 242146_at | 0.0142 | 0.928467 | 0.641019 | 0.621653 | 0.546617 | 0.732431 | SNRPA1 | |
| 204690_at | 0.0142 | 0.957952 | 0.777496 | 0.826562 | 0.835613 | 0.852972 | STX8 | |
| 211806_s_at | 0.0136 | 1.039763 | 1.594321 | 1.397776 | 1.328552 | 1.55341 | KCNJ15 | |
| 209865_at | 0.0136 | 1.047733 | 0.710102 | 0.816724 | 0.792248 | 0.864592 | SLC35A3 | |
| 213742_at | 0.0136 | 1.011462 | 0.679297 | 0.868962 | 0.877236 | 0.787014 | SFRS11 | |
| 240128_at | 0.0136 | 1.107033 | 0.715071 | 0.882091 | 0.944407 | 0.914991 | | |
| 244185_at | 0.0136 | 0.995496 | 0.744312 | 0.801441 | 0.708078 | 0.834261 | METAP2 | ✓ |
| 218967_s_at | 0.0136 | 0.996286 | 0.759103 | 0.961737 | 1.022522 | 1.145249 | PTER | |
| 213546_at | 0.0134 | 1.02815 | 0.820456 | 1.04877 | 0.982193 | 1.187696 | | |
| 223578_x_at | 0.0134 | 1.048886 | 2.950079 | 1.646771 | 2.079696 | 0.590044 | PRO1073 | |
| 230961_at | 0.0134 | 1.038609 | 0.767566 | 0.875764 | 0.953275 | 0.868014 | | |
| 229322_at | 0.0134 | 0.983442 | 0.788516 | 0.753502 | 0.711557 | 0.892672 | PPP2R5E | |
| 212600_s_at | 0.013 | 0.993411 | 0.872366 | 0.974703 | 0.993643 | 1.050638 | UQCRC2 | |
| 215567_at | 0.0125 | 1.009364 | 0.738192 | 0.975616 | 0.943375 | 0.970371 | C14orfl11 | |
| 232304_at | 0.0124 | 0.999634 | 0.652796 | 0.681489 | 0.593741 | 0.920232 | PELI1 | |
| 204351_at | 0.0123 | 0.963259 | 2.371981 | 1.512469 | 1.079271 | 1.213308 | S100P | |
| 206522_at | 0.012 | 0.955883 | 3.010042 | 1.244712 | 0.988527 | 3.348396 | MGAM | |
| 212586_at | 0.0114 | 1.008002 | 0.837131 | 0.874129 | 0.901261 | 1.114442 | CAST | ✓ |
| 208892_s_at | 0.0114 | 0.920758 | 1.564719 | 1.279537 | 1.019926 | 1.621787 | DUSP6 | ✓ |
| 233169_at | 0.011 | 1.033113 | 0.783923 | 0.775127 | 0.613386 | 0.872408 | ZNF350 | |
| 217370_x_at | 0.011 | 0.968191 | 1.400338 | 1.165771 | 1.112696 | 0.933736 | FUS | |
| 219293_s_at | 0.011 | 1.011733 | 0.82505 | 0.905731 | 0.852869 | 0.809294 | GTPBP9 | |
| 224652_at | 0.011 | 0.9961 | 0.716087 | 0.689183 | 0.566871 | 0.902063 | C10orf9 | |
| 239193_at | 0.011 | 1.035104 | 0.682967 | 0.871866 | 0.813023 | 0.929995 | LOC158301 | |
| 235716_at | 0.011 | 0.942661 | 0.604382 | 0.638427 | 0.578708 | 0.785084 | TRA2A | |
| 216560_x_at | 0.0106 | 0.903581 | 3.036922 | 1.792844 | 2.694287 | 1.211703 | IGLC1 | ✓ |
| 236007_at | 0.0105 | 0.997721 | 0.644131 | 1.05134 | 1.177234 | 1.460173 | AKAP10 | |
| 202388_at | 0.0104 | 1.005236 | 1.50306 | 1.286344 | 1.153041 | 1.857699 | RGS2 | ✓ |
| 242290_at | 0.0104 | 1.015474 | 0.714341 | 0.824866 | 0.762161 | 0.825035 | TACC1 | |
| 208893_s_at | 0.0104 | 1.043263 | 1.946543 | 1.514829 | 1.275835 | 2.418984 | DUSP6 | ✓ |
| 219939_s_at | 0.0102 | 1.005175 | 0.78933 | 0.782521 | 0.798766 | 0.911225 | CSDE1 | ✓ |
| 236322_at | 0.0101 | 1.018173 | 0.705105 | 0.780533 | 0.657779 | 0.829348 | FLJ31951 | |
| 228854_at | 0.0101 | 0.953764 | 0.584589 | 0.62531 | 0.58742 | 0.587977 | ZNF145 | |
| 208616_s_at | 0.0101 | 1.005478 | 0.868576 | 0.836087 | 0.784638 | 0.836076 | PTP4A2 | ✓ |
| 201236_s_at | 0.00986 | 0.97503 | 1.349995 | 1.097856 | 1.074564 | 1.387848 | BTG2 | ✓ |
| 208200_at | 0.00947 | 1.055555 | 0.731068 | 1.048517 | 0.900158 | 0.754696 | IL1A | ✓ |
| 243020_at | 0.00898 | 1.018879 | 0.77299 | 0.865276 | 0.869596 | 1.092981 | FAM13A1 | |
| 202431_s_at | 0.00865 | 0.968903 | 1.437987 | 1.087691 | 1.089122 | 1.029774 | MYC | ✓ |
| 243134_at | 0.0086 | 1.045098 | 0.682869 | 0.686464 | 0.582756 | 1.087115 | LOC440309 | |
| 209791_at | 0.0085 | 1.086385 | 1.800922 | 1.190619 | 0.981246 | 1.496994 | PADI2 | |
| 226274_at | 0.00848 | 1.063358 | 0.787488 | 1.088126 | 1.06524 | 1.129191 | LOC158563 | |
| 226489_at | 0.00848 | 1.01411 | 1.439522 | 1.171041 | 1.047809 | 1.250104 | KIAA1145 | |
| 244038_at | 0.00848 | 0.925754 | 1.352068 | 1.40191 | 1.473757 | 1.113687 | LOC112840 | |
| 243788_at | 0.00798 | 0.966688 | 0.591365 | 0.49691 | 0.487266 | 0.608162 | PHF11 | |
| 224341_x_at | 0.00784 | 0.906456 | 1.464877 | 1.187913 | 1.082006 | 1.375654 | TLR4 | ✓ |
| 220710_at | 0.00748 | 1.085801 | 0.621032 | 0.864702 | 0.814153 | 0.694106 | FLJ11722 | |
| 206925_at | 0.00748 | 1.032079 | 1.651645 | 1.109063 | 1.195731 | 1.741701 | ST8SIA4 | ✓ |
| 226315_at | 0.00623 | 1.071869 | 1.380817 | 1.340598 | 1.334991 | 1.140382 | MGC20398 | |
| 242362_at | 0.00601 | 1.008905 | 0.552373 | 0.695976 | 0.675355 | 0.872213 | CUL3 | |
| 217738_at | 0.00601 | 0.970547 | 1.862855 | 1.1219 | 0.836713 | 2.20316 | PBEF1 | ✓ |
| 209193_at | 0.00601 | 0.982949 | 1.341883 | 0.956108 | 0.860525 | 0.967008 | PIM1 | ✓ |
| 212773_s_at | 0.00436 | 0.989453 | 0.794441 | 0.900493 | 1.030907 | 0.800867 | TOMM20 | |
| 223494_at | 0.00436 | 0.996726 | 0.775789 | 0.8442 | 0.837487 | 0.933534 | MGEA5 | |
| 223650_s_at | 0.00403 | 0.981976 | 1.486306 | 1.340921 | 1.447739 | 1.721287 | NRBF2 | |
| 216988_s_at | 0.00403 | 0.987485 | 0.81587 | 0.847148 | 0.813173 | 0.814044 | PTP4A2 | ✓ |
| 219598_s_at | 0.00401 | 1.019 | 0.835652 | 0.94416 | 0.928754 | 0.68444 | RWDD1 | |
| 204308_s_at | 0.00398 | 0.953461 | 1.23924 | 1.141785 | 1.088266 | 1.144755 | KIAA0329 | |
| 215201_at | 0.00398 | 1.005546 | 0.515855 | 0.527043 | 0.639937 | 0.663592 | REPS1 | ✓ |
| 215378_at | 0.00356 | 1.153457 | 0.546117 | 0.896015 | 0.694274 | 0.570313 | ANKHD1 | |
| 203305_at | 0.00326 | 1.024716 | 0.569063 | 0.766591 | 0.784508 | 1.070838 | F13A1 | ✓ |
| 243431_at | 0.00274 | 0.985963 | 0.552993 | 0.672511 | 0.615591 | 0.757856 | BTBD14A | |
| 218559_s_at | 0.00262 | 0.92854 | 1.949402 | 1.17715 | 0.85737 | 2.093452 | MAFB | ✓ |
| 219434_at | 0.00192 | 0.965392 | 1.714783 | 1.33731 | 0.996951 | 1.511987 | TREM1 | ✓ |
| 205220_at | 0.00157 | 0.939424 | 2.391161 | 1.563859 | 1.14199 | 2.094364 | GPR109B | |
| 200976_s_at | 0.00137 | 0.992928 | 0.770992 | 0.946403 | 0.878735 | 1.261498 | TAX1BP1 | ✓ |
| 210772_at | 0.00137 | 0.909041 | 1.758864 | 1.075845 | 1.011895 | 1.804144 | FPRL1 | ✓ |
| 236545_at | 0.00137 | 1.015725 | 0.592298 | 0.58694 | 0.611854 | 0.891015 | FLJ42008 | |
| 206989_s_at | 0.000851 | 1.033835 | 0.75091 | 0.771922 | 0.715956 | 0.881741 | SFRS2IP | |
| 218334_at | 0.000546 | 1.015236 | 0.812377 | 0.916495 | 0.925866 | 0.828603 | THOC7 | |
| 210773_s_at | 0.000546 | 1.093718 | 2.226338 | 1.185403 | 1.177191 | 2.241969 | FPRL1 | ✓ |
| 209864_at | 0.000512 | 0.993217 | 1.651619 | 1.469429 | 1.442434 | 1.386062 | FRAT2 | |
| 202241_at | 0.000512 | 0.976015 | 2.054997 | 1.120879 | 0.83721 | 1.673108 | TRIB1 | |
| 207492_at | 0.000512 | 1.000953 | 0.647088 | 0.653006 | 0.694637 | 0.735727 | NGLY1 | |
| 201739_at | 0.00028 | 0.966253 | 2.072408 | 1.460541 | 1.06163 | 2.05116 | SGK | ✓ |
| 238714_at | 0.000221 | 0.992201 | 0.641299 | 0.87915 | 0.808888 | 0.925665 | RAB12 | |
| 204470_at | 0.000221 | 0.906799 | 2.228335 | 1.26349 | 1.11534 | 1.81416 | CXCL1 | ✓ |
| 202768_at | 0.000221 | 1.76656 | 16.71515 | 6.223381 | 1.674032 | 10.61886 | FOSB | ✓ |
| 243759_at | 0.000221 | 1.005915 | 0.725535 | 0.689206 | 0.635044 | 0.755297 | SFRS15 | |
| 232280_at | 7.88E−05 | 0.973889 | 0.355017 | 0.583071 | 0.531658 | 0.496296 | SLC25A29 | |
| 204748_at | 1.34E−05 | 1.095686 | 4.324824 | 1.973455 | 1.281416 | 4.999675 | PTGS2 | ✓ |
| 205098_at | 1.22E−05 | 1.019905 | 2.191794 | 1.787849 | 1.442583 | 2.212404 | CCR1 | ✓ |
| 39402_at | 1.60E−06 | 1.065533 | 3.577919 | 1.861448 | 1.302511 | 2.931454 | IL1B | ✓ |
| 205067_at | 9.19E−07 | 1.131819 | 4.075302 | 2.399675 | 1.668993 | 3.935437 | IL1B | ✓ |
| 206115_at | 9.19E−07 | 1.006838 | 3.024593 | 1.76009 | 1.368254 | 2.619297 | EGR3 | ✓ |
| 205249_at | 9.19E−07 | 0.935063 | 5.205895 | 3.932818 | 2.526623 | 5.492902 | EGR2 | ✓ |

Flow Cytometric Results. A portion of the PBMCs extracted from each patient was stained with fluorescently labeled antibodies and analyzed by flow cytometry.

No statistically significant differences were found between healthy controls and subjects with newly diagnosed T1D in the absolute number of CD123+ and CD11c+ dendritic cells, basophils, T cells of CD4+/3+, CD8+/3+, or CD8+/3− phenotypes, CD20+/27− naïve B cells, or CD19+/14− B cells. Plasma cell precursors (CD19+/20−) were increased (p=0.02) in new onset T1D patients but not in T2D patients; however this was not statistically significant after correcting for multiple comparisons. One month after T1D diagnosis, the absolute number of plasma cells was not statistically different from that of healthy controls.

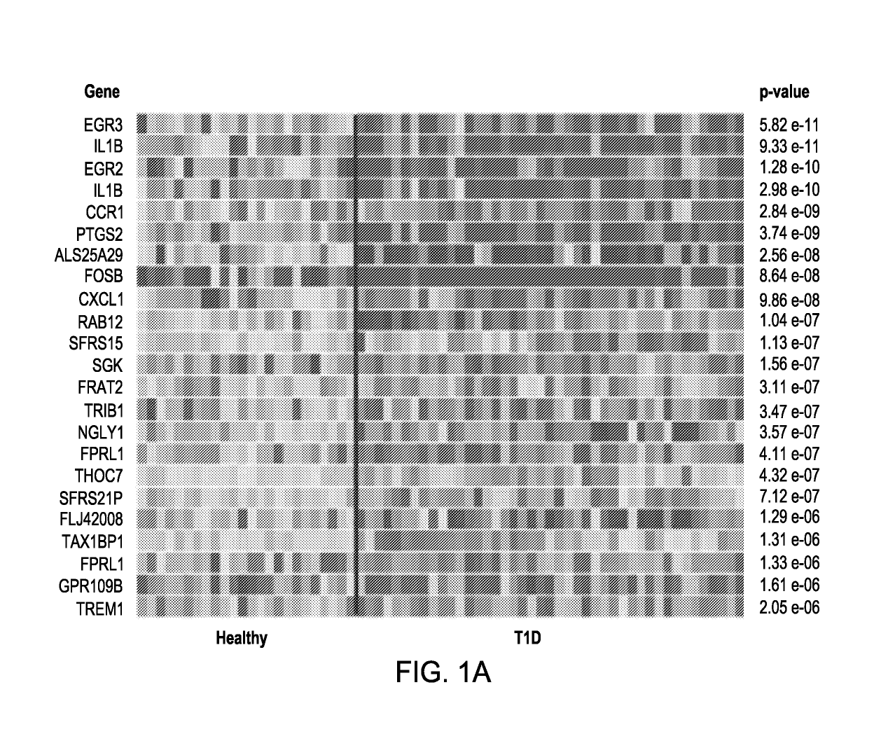

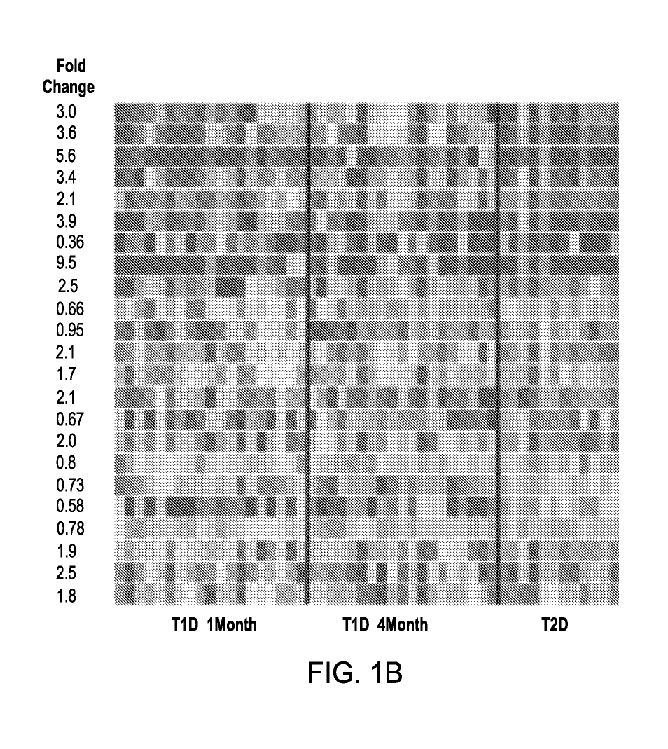

Microarray Results. Of the 44,760 probe sets on the Affymetrix U133A/B chips, 21,514 passed initial quality assurance determined by present flag calls in at least 50% of the subjects in at least one of the cohorts. Data were normalized to the median level of expression of each probe set in the healthy controls. At a false discovery rate (FDR) of 0.05 (corresponding to an uncorrected p value of 0.00072 in this dataset), 312 probe sets representing 282 unique genes differed in expression between new onset T1D patients and healthy controls (Supporting Information, Table 1). An FDR of 0.01 yielded 51 probe sets representing 49 unique genes, and 23 probe sets (21 genes) differed at the stringent Bonferroni-corrected p value of 0.05 (FIG. 1) The most overexpressed genes in T1D patients were interleukin 1 beta (IL1B), early growth response genes 2 (EGR2) and 3 (EGR3), prostaglandin-endoperoxide synthase 2 (PTGS2, COX2), chemokine (C-C motif) receptor 1 (CCR1), and the FOSB oncogene; their expression was increased 2-9 fold over healthy controls. The most significantly underexpressed genes (1.5-3 fold) included RAB12 (a member of the RAS oncogene family), splicing factor, arginine/serine-rich 15 (SFRS15), N-glycanase and solute carrier 25A29 (SLC25A29).

We compared the expression of the most differentially expressed genes at baseline to one and four months after diagnosis. Even with improvement in overall glycemic control (average initial hemoglobin A1c (HbA1c) level of 11.8±2.0% decreased to 7.1±1.3% by four months), EGR2 remained overexpressed in patients (p=0.0006 at 4 month follow-up versus healthy controls) at four months after diagnosis whereas EGR3, IL1B, CCR1, and FOSB decreased toward healthy control levels (Supporting Information, FIG. 1). RAB12, SFRS15, NGLY1 and SLC25A29 remained underexpressed throughout the study period.

We also compared profiles of 12 patients with newly diagnosed, poorly controlled T2D to the newly diagnosed T1D patients. Eighteen of the 21 most highly differentially expressed genes in newly diagnosed T1D were similarly regulated in T2D (FIG. 1).

Genes known to be specifically expressed in plasma cells (such as immunoglobulin genes) were generally more highly expressed in T1D patients than in controls or T2D patients; of 76 genes associated with plasma cells (Chaussabel et al, unpublished observations), 57 (75%) were overexpressed with uncorrected p values<0.05 using Mann-Whitney U statistical group comparisons. To determine whether the overexpression of plasma cell-specific genes reflected increased gene expression within plasma cells or increased cell number, we averaged the normalized data from each patient for the 76 genes associated with plasma cells and compared this value with the absolute number of plasma cells. Mean expression for the 76 plasma cell genes generated from array data was correlated with a Spearman r of 0.53 (95% confidence interval, 0.30-0.71) and two-tailed p value<0.0001 to absolute plasma cell numbers determined from flow cytometry. There was no correlation between the number or titer of positive autoantibodies and expression of plasma cell genes.

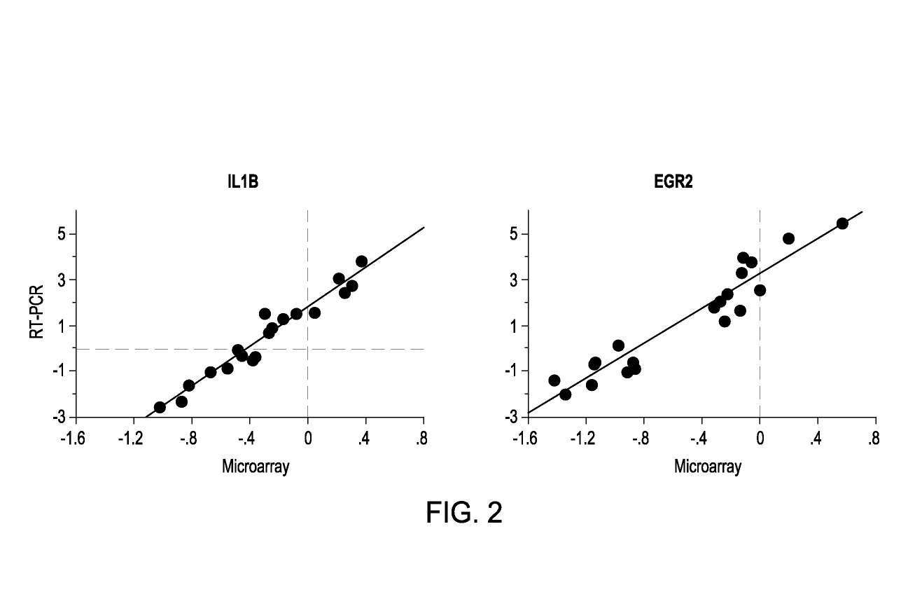

RT-PCR. To confirm selected microarray results using an independent technique, normalized microarray values were compared to delta C_{T} values for the same genes obtained from RT-PCR studies. Spearman r values ranged from 0.62 to 0.94 for six genes (FIG. 2) with p values ranging from 0.0031 to <0.0001.

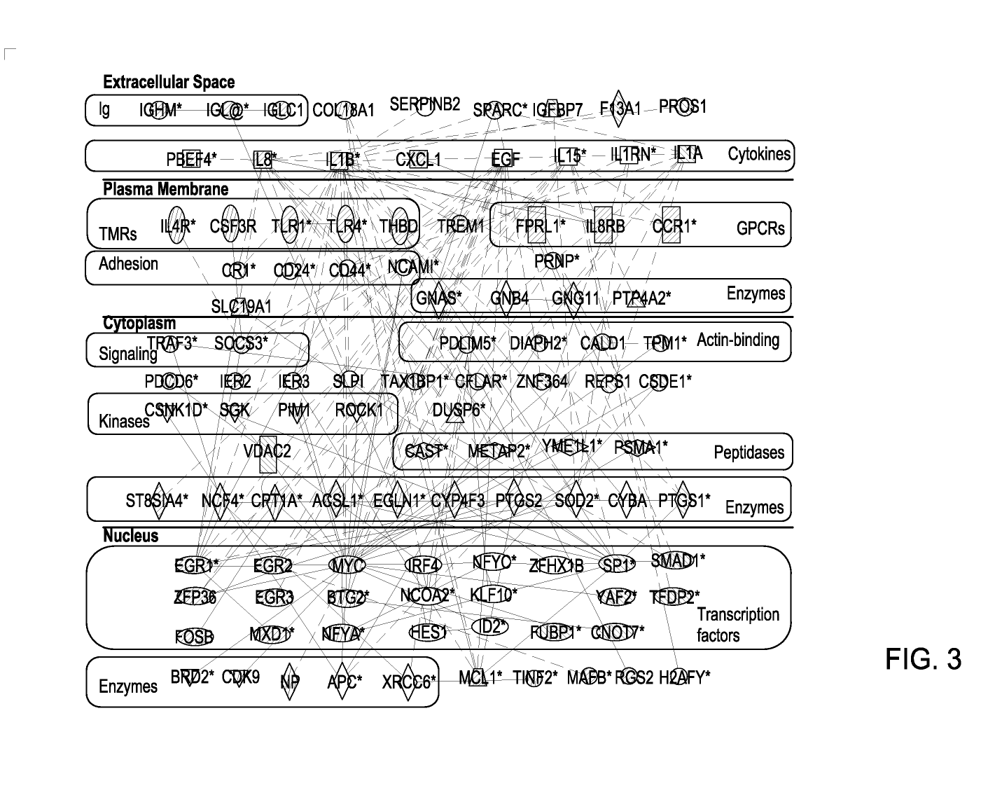

Pathway analysis. To identify functional relationships between differentially-expressed genes, we used a predefined knowledge base containing over 10,000 curated human genes(14). Of the 21,514 defined as ‘present’ on the arrays, 5897 genes had entries within the knowledge base. When an FDR of 0.05 was used as a threshold criterion (282 genes differentially expressed between new T1D patients and healthy controls), 11 partially-overlapping sub-networks were identified that were enriched for these genes. The top-scoring sub-network included 35 genes meeting the threshold criterion with a probability of 10⁻⁶¹ that the curated interrelationships between these genes occurred by chance. This network was extended by merging all overlapping networks. Genes within these networks that did not meet the threshold FDR of 0.05 were retained if they were nevertheless differentially expressed with an uncorrected p value of 0.05. The result was a network of 103 genes with a probability score of 10⁻⁹³. This network preferentially included the most differentially-expressed genes; whereas 81/282 genes in the input dataset that differed at an FDR of 0.05 were included in this network, 22/49 that differed at an FDR of 0.01 were included, and 11/21 genes that differed at a Bonferroni-corrected p value of 0.05 were included (p=0.01 by chi-square for the differing proportions of genes included in the network at the different threshold values). There were 222 connections (i.e., known relationships) between the genes in this network (FIGS. 2 and 3).

To identify groups of genes within this network that were differentially expressed in a manner unique to T1D, we compared levels of expression in T1D to those seen in T2D patients, identifying 47/103 genes that differed between T1D and T2D at an FDR of 0.05. These genes tended not to be distributed randomly within the network, as illustrated by inspecting the two most highly connected genes in the network, IL1B and MYC (36 connections each). IL1B is similarly overexpressed in T1D and T2D patients. In contrast, MYC is overexpressed only in T1D patients; thus, it differs significantly in expression between T1D and T2D patients. When the 10 genes that are connected in the network to both IL1B and MYC were excluded, 16/26 genes connected to MYC, but only 9/26 genes connected to IL1B, differed in expression between T1D and T2D (p=0.05, Fisher's Exact Test) (FIG. 2).

The cellular functions most strongly associated with this network (Table 2) include cell death (51 genes, p<5×10⁻¹⁸) and cell proliferation (50 genes, p<10⁻¹³). Excluding genes connected to both IL1B and MYC, genes connected to IL1B were more likely to have functions associated with proliferation (19/26) than genes connected to MYC (7/26, p=0.002, Fisher's Exact Test) whereas genes associated with apoptosis were equally likely to be connected to IL1B or MYC (14/26 versus 12/26, respectively).

TABLE 2
Cellular functions associated with type 1 diabetes
based on Ingenuity pathways.

| Function | P value | Number of genes |
|---|---|---|
| Apoptosis of eukaryotic cells | 4.74E−18 | 51 |
| Proliferation of cells | 9.52E−14 | 50 |
| Development of lymphatic system cells | 2.05E−13 | 20 |
| Quantity of cells | 1.28E−12 | 36 |
| Cell death of tumor cell lines | 1.37E−12 | 34 |
| Hematopoiesis | 1.60E−12 | 25 |
| Quantity of lymphatic system cells | 3.48E−10 | 22 |
| Quantity of leukocytes | 5.42E−10 | 21 |
| Production of prostaglandin E2 | 1.90E−9 | 9 |
| Inflammatory response | 2.72E−9 | 19 |

With >40,000 probe sets, whole-genome microarray studies are liable to type 1 errors due to simultaneously testing of multiple hypotheses. The most frequently used method of controlling the type I error rate while maintaining adequate power (controlling the type II error rate) is the FDR (15, 16), the expected proportion of truly null hypotheses among all the rejected null hypotheses. In some studies, this is balanced by concurrent consideration of false negative rates (17).

A powerful alternative strategy consists of testing for differences in expression of predefined clusters or networks of genes rather than individual genes, thus drastically reducing the number of tested hypotheses. We used such an approach to delineate consistent similarities and differences in gene expression between T1D and T2D patients. Most (51/81) of the differentially-expressed genes in the network have no prior reported associations with diabetes, diabetes complications, or hyperglycemia.

IL1B is overexpressed in patients with both forms of diabetes, whereas MYC is overexpressed only in T1D patients. More genes differing in expression between T1D and T2D are connected in the network to MYC than to IL1B. These findings suggest that T1D and T2D have some pathogenetic mechanisms in common (exemplified by overexpression of IL1B) despite their distinct underlying etiologies (evidenced by overexpression of MYC only in T1D patients).

Changes in gene expression common to type 1 and type 2 diabetes. IL-1β has previously been implicated in the pathogenesis of diabetes (18, 19). Patients with either form of diabetes are hyperglycemic at diagnosis. IL-1β is induced in monocytes in vitro by high glucose levels (20). Incubation of human or animal islets or insulinoma cell lines with IL-1β (along with TNFα and/or interferon-gamma in many studies) inhibits insulin secretion and leads to apoptosis of beta cells (21). Of genes connected to IL1B in the network, the most evidence for dysregulation in diabetes exists for PTGS2 (COX2), which is increased in mononuclear cells from established diabetic patients (20, 22) and is also upregulated in vitro by high glucose concentrations (20).

It is instructive to compare diabetes to a disease in which IL-1β is known to play a pathogenetic role, juvenile idiopathic arthritis of systemic onset (SOJIA). There is a median 1.7-fold increase in IL1B expression in SOJIA PBMCs versus healthy controls (23), compared with a >3 fold median increase in newly diagnosed T1D patients. Of the top 10 mostly highly overexpressed genes in T1D patients, five—IL1B, EGR3, PTGS2, CCR1 and CXCL1—are also overexpressed in SOJIA patients and/or are overexpressed when healthy PBMCs are incubated with SOJIA serum (23). Although our data suggest the importance of IL1B dysregulation in diabetes as well as SOJIA, diabetes is obviously not the sole result of IL-1β secretion since patients with diabetes do not have systemic effects of IL-1β-mediated inflammation such as fever and arthritis.

It has been suggested that T1D and T2D share a final common pathway for beta cell dysfunction: hyperglycemia in pancreatic islets upregulates IL1B, leading to beta cell dysfunction and further hyperglycemia (5, 24). However, hyperglycemia has not been consistently documented to affect IL-1β secretion by beta cells (25). The present study refines the idea of a final common pathway to include immune effector cells: beta cell dysfunction leads to hyperglycemia, increasing inflammation (including secretion of IL-1β and prostaglandins by immune effector cells), thus exacerbating beta cell dysfunction, and causing more hyperglycemia.

The mechanisms by which hyperglycemia increases IL1B expression in PBMCs remain to be determined. Perhaps protein glycation resulting from chronic hyperglycemia increases IL-1β levels. Advanced glycation endproducts (AGEs) interact with the receptor for advanced glycation endproducts (RAGE) and trigger release of IL-1β from monocytes in some (26) but not all studies (27). The involvement of relatively long-lived AGEs could explain why many of the changes in the present study persisted for several months after insulin treatment was initiated.

Changes in gene expression specific for type 1 diabetes. Although dysregulation of MYC has not been previously reported in human diabetes, it is overexpressed in peripheral leukocytes of diabetes-prone non-obese diabetic (NOD) mice, relative to control C57BL6 mice, before development of diabetes (28). Transgenic mice in which MYC is overexpressed in pancreatic beta cells develop neonatal diabetes with increased islet hyperplasia accompanied by a marked increase in apoptosis and decreased insulin gene expression (29). The present results support and extend these findings by demonstrating increased expression of MYC in peripheral leukocytes at diagnosis of T1D, and associated dysregulation of many genes implicated in apoptosis. Some of these changes are not seen in T2D patients with similar levels of hyperglycemia but persist for at least 4 months after T1D diagnosis. Therefore, changes in expression of MYC and associated genes are not a simple response to hyperglycemia. Whether the changes affect quantity or functioning of immune effectors, or reflect correspondingly dysregulated gene expression within pancreatic beta cells, cannot yet be determined.

We documented increased numbers of plasma cell precursors at diagnosis (albeit at a p value that was not significant after correcting for multiple comparisons), increased expression of plasma cell-specific genes such as immunoglobulins, and a significant correlation between these findings. Although T1D is considered to result primarily from the actions of T cells, it is increasingly recognized that B cells may play a role as well. Eliminating maternal antibodies in non-obese diabetic (NOD) mice abrogates the development of diabetes in susceptible offspring (30). This may be a consequence of cell-surface immunoglobulins on B cells functioning in antigen presentation (31). The importance of B cells in the development of diabetes in humans is now being studied in a therapeutic trial of rituximab (anti-CD20, which targets B cells) in patients with new-onset T1D (32).

Peripheral blood mononuclear cells (PBMCs) were samples rather than pancreatic islets. Although islet-infiltrating immune cells are presumably in equilibrium with circulating pools, they are diluted in the circulation. Similarly, changes in gene expression that are confined to a particular cell type may be difficult to detect in unfractionated PBMCs (33). Nevertheless, PBMCs reflect generalized abnormalities in immune regulation as well as systemic effects of the metabolic derangements of untreated diabetes. It is possible that many of the observed changes are directly or indirectly the consequence of chronic hyperglycemia. While many such changes may be accompanied by parallel changes in pancreatic beta cells, it will be difficult to definitively answer this question due to the inaccessibility of the pancreas in newly diagnosed T1D patients.

Second, the Ingenuity knowledge base, although extensive, is incomplete with regard to interrelationships between genes (i.e., the analysis is subject to literature biases), and conversely, many of those relationships are of uncertain functional significance or may be irrelevant in PBMCs.

Third, we studied patients with new-onset diabetes. Key events may have run their course by the time hyperglycemia supervenes. We found no evidence of interferon-gamma or tumor necrosis factor-α overexpression in PBMCs from newly-diagnosed T1D patients, yet many studies implicate both of these cytokines in diabetes pathogenesis. Perhaps they are involved in human T1D earlier in the course of the disease, but differences between animal models of T1D and humans might also account for this discrepancy.

Therapeutic implications. Although the abnormalities in PBMCs in new onset T1D patients become less prominent over the first few months of insulin therapy, further damage to beta cells is occurring during this time. Thus the present results imply that disease-modifying interventions should be initiated as quickly as possible after diagnosis. The observation that many of the observed changes in gene expression resolve with insulin therapy provides a rationale for the beneficial effects of aggressive glycemic control early in the disease in preserving residual beta cell function(34). Our results also suggest several promising therapeutic targets. The elevation in plasma cells could be treated by attacking precursor B cells, and as mentioned, a trial of rituximab (anti-CD20) is already underway. Elevated expression of PTGS2 (and thus, presumably, high prostaglandin levels) could be treated with non-steroidal anti-inflammatory agents; sodium salicylate was first suggested as a treatment for diabetes in the 19^{th} century(35). The marked elevation in IL1B expression could be treated with anakinra (IL-1 receptor antagonist protein), which has proven highly effective in SOJIA (23). Blockers of chemokine receptors including CCR1 have reached phase 2 clinical trials as anti-inflammatory agents(36). In addition to providing rationales for therapeutic interventions, abnormalities detected in the present study might ultimately provide useful biomarkers for the efficacy of disease-modifying interventions

Materials and Methods.

Subjects. The study was approved by the Institutional Review Boards of UT Southwestern Medical Center and Baylor Institute for Immunology Research. Informed consent was obtained from parents or legal guardians and informed assent was obtained from patients aged 10 years and older.

Patients between the ages of two and eighteen years with newly diagnosed T1D by American Diabetes Association (ADA) criteria(37) and healthy controls were eligible if they weighed greater than 20 kg. Patients with T2D as defined by ADA criteria(37) were required to have HbA1c levels of ≧8% so as to be matched biochemically to the T1D patients. Patients were excluded from the study if they had an active or presumed infection, other autoimmune disease, were pregnant, were taking immune modulators, or had an initial hematocrit less than 27%. Patients were also excluded if it was uncertain whether they had T1D or T2D.

Processing of blood samples. Blood samples were collected in EDTA tubes. Initial samples were obtained after diabetic ketoacidosis (if present) had resolved, within five days (but usually within 2-3 days) of diagnosis. Peripheral blood mononuclear cells (PBMCs) were isolated using Ficoll gradients within 4 hours of each blood draw; if not processed immediately, cells were lysed in RLT lysis buffer containing β-mercaptoethanol and stored at −80° C. (Qiagen, Valencia, Calif.). Serum samples were also frozen at −80° C. Total RNA was extracted using the RNeasy® Mini Kit according to the manufacturer's protocol (Qiagen, Valencia, Calif.). RNA integrity was assessed using an Agilent 2100 Bioanalyzer (Agilent, Palo Alto, Calif.).

Autoantibody testing. Serum samples were tested for antibodies to insulin, IA-2 and GAD65, using ELISA kits from Kronus Inc. (Boise, Id.) at either ARUP (Salt Lake City, Utah) or in the laboratory of Phillip Raskin, M.D., UT Southwestern Medical Center (Dallas, Tex.).

Flow cytometry. PBMCs from each sample were analyzed by flow cytometry (FACSCalibur, BD Biosciences). We used antibodies against CD3, CD14, CD19 and CD16 (Becton-Dickinson, Franklin Lakes, N.J., USA) in one well to differentiate between B cells, T cells, monocytes and natural killer cells. Anti-CD3, CD14, CD8 and CD4 antibodies differentiated between cytotoxic and helper T cells and monocytes. Anti-lineage FITC cocktail, and anti-CD 123, HLA DR and CD11c antibodies differentiated between the various types of dendritic cells whereas anti-CD27, CD138, CD20 and CD19 antibodies distinguished naïve, memory B cells and plasma cell precursors. Studies were analyzed after gating on live cells according to forward side scatter/side light scatter. A minimum of 100,000 cells was used for each staining condition, and 5,000-50,000 events were recorded for analysis.

Microarray assays. From 2-5 μg of total RNA, double-stranded cDNA containing the T7-dT(24) promoter sequence was generated using GeneChip® One-Cycle cDNA Synthesis Kit (Invitrogen, Santa Clara, Calif.). This cDNA was used as a template for in vitro transcription single round amplification with biotin labels using the GeneChip® IVT Labeling Kit (from Affymetrix Inc, Santa Clara, Calif.). Biotinylated cRNA targets were purified using the Sample Cleanup Module (Affymetrix) and subsequently hybridized to human U133A and U133B GeneChips (Affymetrix Inc, Santa Clara, Calif.) according to the manufacturer's protocols. Affymetrix GeneChips contain 44,760 probe sets, represented by ten to twenty unique probe pairs, allow detection of different genes probes and expressed sequence tags (ESTs). Arrays were scanned using a laser confocal scanner (Agilent). Any artifacts were masked out so that the affected probe cells were not used in the analyses. Samples with excessive background noise or poor cRNA quality based on internal control genes, actin or GAPDH were not used in the analysis.

RT-PCR. 2 μg cRNA samples were converted to cDNA using TaqMan® Reverse Transcription Reagents and a 2720 Thermocycler (Applied Biosystems, Foster City, Calif.). Quantitative Real-Time PCR was performed using 50 ng of selected targets, in duplicate, using pre-developed primers and probe TaqMan® Gene Expression Assays (Applied Biosystems, Foster City, Calif.) on the ABI Prism 7900HT Sequence Detection System. Data were analyzed (SDS2.3) using the relative comparative cycle-threshold method (C_{T}) with hypoxanthine ribosyl transferase (huHPRT) as the endogenous control for each target confirmed. Samples from 7 healthy controls, 14 T1D patients and 3 T2D patients were analyzed. Delta C_{T} values were compared to the negative log of normalized microarray expression data.

Statistical analysis. For each Affymetrix U133A or U133B Gene Chip,® raw intensity data were normalized to the mean intensity of all measurements on that chip and scaled to a target intensity value of 500 in GeneChip Operating System version 1.0. With use of Genespring software, version 7.3.1, the value for each gene in each patient sample array was divided by the median of that gene's measurement from the cohort of healthy volunteers. A filter was applied based on Affymetrix flag calls: probe sets were selected if “Present” in at least 50% of samples in either group (healthy controls or patients). Class comparisons were performed using parametric tests after log transformation.

To identify functional relationships between differentially-expressed genes, we used a predefined knowledge base containing over 10,000 curated human genes and a large predefined network of interrelationships between these genes(14) (Ingenuity Systems, Redwood City, Calif.). Normalized expression values and p values from the entire array study were entered along with a threshold value for statistical significance, a Benjamini-Hochberg false discovery rate (FDR) of 0.05(15, 16). The database returned portions of the predefined network containing up to 35 genes each that were optimized for the number of genes exceeding the threshold. P values for these sub-networks were calculated by Fisher's exact tests, and overlapping networks were merged. Additionally, p values were calculated for the numbers of genes having known functions in specified categories.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Atkinson, M. A. & Maclaren, N. K. (1994) N Engl J Med 331, 1428-36.

2. Bach, J. F. (1994) Endocr Rev 15, 516-42.

3. Rabinovitch, A. (2003) Adv Exp Med Biol 520, 159-93.

4. Hohmeier, H. E., Tran, V. V., Chen, G., Gasa, R. & Newgard, C. B. (2003) Int J Obes Relat Metab Disord 27 Suppl 3, S12-6.

5. Donath, M. Y., Storling, J., Maedler, K. & Mandrup-Poulsen, T. (2003) J Mol Med 81, 455-70.

6. Mathis, D., Vence, L. & Benoist, C. (2001) Nature 414, 792-8.

7. Basu, S., Larsson, A., Vessby, J., Vessby, B. & Berne, C. (2005) Diabetes Care 28, 1371-5.

8. Shimabukuro, M., Koyama, K., Lee, Y. & Unger, R. H. (1997) J Clin Invest 100, 1750-4.

9. Green, E. A. & Flavell, R. A. (2000) Immunity 12, 459-69.

10. Guerder, S., Picarella, D. E., Linsley, P. S. & Flavell, R. A. (1994) Proc Natl Acad Sci USA 91, 5138-42.

11. Tanaka, Y., Asakawa, T., Asagiri, K., Akiyoshi, K., Hikida, S. & Mizote, H. (2004) Kurume Med J 51, 99-103.

12. Felner, E. I. & White, P. C. (2001) Pediatrics 108, 735-40.

13. Umpaichitra, V., Banerji, M. A. & Castells, S. (2002) J Pediatr Endocrinol Metab 15 Suppl 1, 525-30.

14. Calvano, S. E., Xiao, W., Richards, D. R., Felciano, R. M., Baker, H. V., Cho, R. J., Chen, R. O., Brownstein, B. H., Cobb, J. P., Tschoeke, S. K., Miller-Graziano, C., Moldawer, L. L., Mindrinos, M. N., Davis, R. W., Tompkins, R. G. & Lowry, S. F. (2005) Nature 437, 1032-7.

15. Jung S H, J. W. (2006) Bioinformatics 22, 1730-6.

16. Reiner A, Y. D., Benjamini Y (2003) Bioinformatics 19, 368-75.

17. Norris, A. W. & Kahn, C. R. (2006) Proc Natl Acad Sci USA 103, 649-53.

18. Mandrup-Poulsen, T. (1996) Diabetologia 39, 1005-29.

19. Bergholdt, R., Heding, P., Nielsen, K., Nolsoe, R., Sparre, T., Storling, J., Nerup, J., Pociot, F. & Mandrup-Poulsen, T. (2004) Adv Exp Med Biol 552, 129-53.

20. Shanmugam, N., Gaw Gonzalo, I. T. & Natarajan, R. (2004) Diabetes 53, 795-802.

21. Mandrup-Poulsen, T. (2001) Diabetes 50 Suppl 1, S58-63.

22. Litherland, S. A., She, J. X., Schatz, D., Fuller, K., Hutson, A. D., Peng, R. H., Li, Y., Grebe, K. M., Whittaker, D. S., Bahjat, K., Hopkins, D., Fang, Q., Spies, P. D., North, K., Wasserfall, C., Cook, R., Dennis, M. A., Crockett, S., Sleasman, J., Kocher, J., Muir, A., Silverstein, J., Atkinson, M. & Clare-Salzler, M. J. (2003) Pediatr Diabetes 4, 10-8.

23. Pascual, V., Allantaz, F., Arce, E., Punaro, M. & Banchereau, J. (2005) J Exp Med 201, 1479-86.

24. Maedler, K., Sergeev, P., Ris, F., Oberholzer, J., Joller-Jemelka, H. I., Spinas, G. A., Kaiser, N., Halban, P. A. & Donath, M. Y. (2002) J Clin Invest 110, 851-60.

25. Welsh, N., Cnop, M., Kharroubi, I., Bugliani, M., Lupi, R., Marchetti, P. & Eizirik, D. L. (2005) Diabetes 54, 3238-44.

26. Shanmugam N, K. Y., Lanting L, Natarajan R (2003) J Biol Chem 278, 34834-44.

27. Valencia J V, M. M., Koehne C, Rediske J, Hughes T E (2004) Diabetologia 47, 844-52.

28. Gerling, I. C., Singh, S., Lenchik, N. I., Marshall, D. R. & Wu, J. (2006) Mol Cell Proteomics 5, 293-305.

29. Laybutt, D. R., Weir, G. C., Kaneto, H., Lebet, J., Palmiter, R. D., Sharma, A. & Bonner-Weir, S. (2002) Diabetes 51, 1793-804.

30. Greeley, S. A., Katsumata, M., Yu, L., Eisenbarth, G. S., Moore, D. J., Goodarzi, H., Barker, C. F., Naji, A. & Noorchashm, H. (2002) Nat Med 8, 399-402.

31. Wong, F. S., Wen, L., Tang, M., Ramanathan, M., Visintin, I., Daugherty, J., Hannum, L. G., Janeway, C. A., Jr. & Shlomchik, M. J. (2004) Diabetes 53, 2581-7.

32. http://www.diabetestrialnet.org

33. Laudanski, K., Miller-Graziano, C., Xiao, W., Mindrinos, M. N., Richards, D. R., De, A., Moldawer, L. L., Maier, R. V., Bankey, P., Baker, H. V., Brownstein, B. H., Cobb, J. P., Calvano, S. E., Davis, R. W. & Tompkins, R. G. (2006) Proc Natl Acad Sci USA 103, 15564-9.

34. Shah, S. C., Malone, J. I. & Simpson, N. E. (1989) N Engl J Med 320, 550-4.

35. Williamson, R. T. (1901) Br Med J. 1, 760-762.

36. Ribeiro, S. & Horuk, R. (2005) Pharmacol Ther 107, 44-58.

37. (2007) Diabetes Care 30 Suppl 1, S42-7.

What is claimed is:

1. A method for diagnosing, preventing or treating a subject suspected of having Type 1 diabetes comprising:
determining the level of gene expression in peripheral blood mononuclear cells of one or more genes from the group of genes in Table I; and
providing the subject with IL-1β antagonists if the subject have elevated levels of IL-1β gene expression.

2. The method of claim 1, wherein the IL-1β antagonist comprises anakinra, an anti-IL-1β siRNA, anti-IL-1β.

3. The method of claim 1, wherein the IL-1β antagonist is further encapsulated in a capsule, caplet, softgel, gelcap, suppository, film, granule, gum, insert, pastille, pellet, troche, lozenge, disk, poultice or wafer.

4. The method of claim 1, wherein IL-1β antagonist is a pharmaceutical composition adapted for administration via parenteral, intravenous, oral, intramuscular, intraaortal, intrahepatic, intragastric, intranasal, intrapulmonary, intraperitoneal, subcutaneous, rectal, vaginal, intraosseal or dermal delivery.

5. A method of identifying a human subject suspected of having diabetes comprising determining the expression level of a biomarker comprising one or more of the following genes: interleukin-1β (IL1B), early growth response gene 3 (EGR3), prostaglandin-endoperoxide synthase 2 (PTGS2) and combinations thereof.

6. The method of claim 5, wherein the step of determining expression levels is performed by measuring amounts of mRNA, protein and combinations thereof.

7. The method of claim 5, wherein the step of determining expression levels is performed using hybridization of nucleic acids on a solid support, an oligonucleotide array, sequencing and combinations thereof.

8. The method of claim 5, wherein the step of determining expression levels is performed using cDNA which is made using mRNA collected from the human cells as a template.

9. The method of claim 5, wherein the biomarker comprises mRNA level and is quantitated by a method selected from the group consisting of polymerase chain reaction, real time polymerase chain reaction, reverse transcriptase polymerase chain reaction, hybridization, probe hybridization, and gene expression array.

10. The method of claim 5, wherein the step of determining the level of expression is accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing.

11. The method of claim 5, wherein the sample comprises a peripheral blood mononuclear cell.

12. A method of identifying a human subject suspected of having Type 1 diabetes comprising determining the expression level of a biomarker comprising one or more of the following genes: interleukin-1β (IL1B), early growth response gene 3 (EGR3), and prostaglandin-endoperoxide synthase 2 (PTGS2).

13. The method of claim 12, wherein the step of determining expression levels is performed by measuring amounts of mRNA, protein and combinations thereof.

14. The method of claim 12, wherein the step of determining expression levels is performed using hybridization of nucleic acids on a solid support, an oligonucleotide array, sequencing and combinations thereof.

15. The method of claim 12, wherein the step of determining expression levels is performed using cDNA which is made using mRNA collected from the human cells as a template.

16. The method of claim 12, wherein the biomarker comprises mRNA level and is quantitated by a method selected from the group consisting of polymerase chain reaction, real time polymerase chain reaction, reverse transcriptase polymerase chain reaction, hybridization, probe hybridization, and gene expression array.

17. The method of claim 12, wherein the step of determining the level of expression is accomplished using at least one technique selected from the group consisting of polymerase chain reaction, heteroduplex analysis, single stand conformational polymorphism analysis, ligase chain reaction, comparative genome hybridization, Southern blotting, Northern blotting, Western blotting, enzyme-linked immunosorbent assay, fluorescent resonance energy-transfer and sequencing.

18. The method of claim 12, wherein the sample comprises a peripheral blood mononuclear cell.

19. A computer implemented method for determining a Type 1 diabetes phenotype in a sample comprising:
obtaining one or more probe intensities for one or more genes listed in Table 1 from a sample;
diagnosing the Type 1 diabetes based upon an increase in the probe intensities for the one or more genes as compared to normal gene expression, expression of genes from a non-Type 1 diabetic patient, a Type 3 diabetic patient and combinations thereof.

20. A computer readable medium comprising computer-executable instructions in a system for performing the method for diagnosing a patient with Type 1 diabetes comprising:
diagnosing Type 1 diabetes based upon the sample probe intensities for six or more genes selected those genes listed in Table 1 and combinations thereof; and
calculating a linear correlation coefficient between the sample probe intensities and reference probe intensities; and accepting the tentative diagnosis of Type 1 diabetes if the linear correlation coefficient is greater than a threshold value.

21. The system of claim 20, wherein the biomarkers are selected from the genes for interleukin-1β (IL1B), early growth response gene 3 (EGR3), and prostaglandin-endoperoxide synthase 2 (PTGS2) and combinations thereof in peripheral blood mononuclear cells.

22. A method for treating a subject suspected of having Type 1 diabetes comprising providing the subject with a therapeutically effective amount of one or more IL-1β antagonists sufficient to spare pancreatic beta cells.

23. The method of claim 22, wherein the IL-1β antagonist comprises anakinra, an anti-IL-1β siRNA, anti-IL-1β.

24. The method of claim 22, wherein the IL-1β antagonist is further encapsulated in a capsule, caplet, softgel, gelcap, suppository, film, granule, gum, insert, pastille, pellet, troche, lozenge, disk, poultice or wafer.

25. The method of claim 22, wherein IL-1β antagonist is a pharmaceutical composition adapted for administration via parenteral, intravenous, oral, intramuscular, intraaortal, intrahepatic, intragastric, intranasal, intrapulmonary, intraperitoneal, subcutaneous, rectal, vaginal, intraosseal or dermal delivery.

26. A pharmaceutical composition for treating a subject suspected of having Type 1 diabetes comprising a therapeutically effective amount of one or more IL-1β antagonists sufficient to spare pancreatic beta cells.

27. The composition of claim 26, wherein the IL-1β antagonist comprises anakinra, an anti-IL-1β siRNA, anti-IL-1β.

28. The composition of claim 26, wherein the IL-1β antagonist is further encapsulated in a capsule, caplet, softgel, gelcap, suppository, film, granule, gum, insert, pastille, pellet, troche, lozenge, disk, poultice or wafer.

29. The composition of claim 26, wherein IL-1β antagonist is a pharmaceutical composition adapted for administration via parenteral, intravenous, oral, intramuscular, intraaortal, intrahepatic, intragastric, intranasal, intrapulmonary, intraperitoneal, subcutaneous, rectal, vaginal, intraosseal or dermal delivery.
